(19) United States
(12) Patent Application Publication
(10) Pub. No.: US 2019/0000594 A1
(43) Pub. Date: Jan. 3, 2019

(54) DENTAL KIT

(71) Applicant: DENTICARE IMPLANTOLOGY CENTER S.R.L., Bucharest (RO)

(72) Inventor: Bogdan Constantin VLADILA, Bucharest (RO)

(21) Appl. No.: 15/756,082

(22) PCT Filed: Aug. 24, 2016
(86) PCT No.: PCT/RO2016/000024

§ 371 Date: Feb. 28, 2018

(30) Foreign Application Priority Data
Sep. 1, 2015 (RO) A 2015 00629

(51) Int. Cl. A61C 8/02; A61N 2/00; A61N 2/02; A61C 19/06

(52) U.S. Cl. CPC A61C 8/0007; A61C 19/06; A61N 2/02; A61N 2/004

(57) ABSTRACT

The invention relates to a dental kit used for accelerating the recovery of the tissues affected by the insertion of an implant, and, implicitly, the osteointegration thereof, and also for treating diseases in the oral-maxillofacial area, such as periodontosis. According to the invention, the dental kit comprises two external mouth guards (A and B), the first of which has three work-points, and the second one has two work-points, an external mouth guard (F) with multiple work-points, a material (12) consisting of a polymer with high polarity and an apparatus (D) generating an electromagnetic field which generates, inside the mouth guards (A or B or F), a uniform field with an induction ranging between 0.7 . . . 0.9 mT, with a variation ranging between 5 . . . 10%, in the area subjected to the electromagnetic field, on a distance of 1 cm around the same point, and on a volume as high as possible in the areas where the tooth root or the implant is to be positioned, produced by a current of 200 mA, with a frequency ranging between 7 . . . 8 Hz, preferably 7.69 Hz, the field lines being perpendicular on the target-tissue.

The present invention relates to a dental kit used for increasing tissue regeneration affected by the insertion of an implant, and thereby its osteintegration, but also for the treatment of some diseases from an orromaxillofacial area such as periodontitis.

In many varied fields of orthopedic procedures, it is of decisive importance lowering the period of healing of fractured bones and damaged tissues, as well as of the biointegration of implants.

The resorbtion of tissue and bone structure, affected by an implant or otherwise, is a continuous process which takes time. The reason is a lowering of the local blood microcirculation and, in the particular field of dentistry for instance, results in dental mobility. The level of tissue integration depends firstly on the development rate of new cells in the region of surgical trauma. If the flow of these cells is high, a rapid revascularization of the region is produced and the cells can survive on the implanted material. The protoglycans level may be reduced and the proportion of direct contact between bone and implant will increase in percentage. Normally, dead cells are immediately replaced by new progenitor cells, to be converted into osteoblast, cementoblast cells, etc, according to the specific character of that region.

Various technical-medical solutions have been developed for improving the biointegration of implants. The use in processing the implants of components made of porous titanium, of zirconium oxide, the alloy of titanium with zirconium, are only a few known solutions for improving biocompatibility. Continuous research and development both in connected and in far removed fields of application have opened surprising ways of further improvement in cellular regeneration.

In 1952, Doctor Winfried Otto Schumann of the Faculty of Science in Munich succeeded in demonstrating the theory that the terrestrial space comprised between the earth surface and ionosphere behaves as a wave guide and at the same time as a resonance box. The frequency spectrum of this space ranges approximately between 6 and 50 Hz, with the main average value of 7.83 Hz, as life on earth was created within this space, all that we call a living organism was adapted to this frequency. Vital functions of organisms deteriorate when not subjected to this frequency over extended periods of time. This explains why astronauts' bodies suffer important disturbances during extra-terrestrial flight, such as parodonthopathy and osteoporosis. These conditions have been at least partly prevented by artificially generating the 7.83 Hz frequency on board spaceships. Scientists have discovered that, besides the basic frequency vital for each organism to function, the internal organs and cells constituting them also react favorably to other frequencies, some of them vital for their restoration when they have been subjected to lesions or when suffering from certain affections.

Further, James Oschman demonstrated that each normal or pathological event carried on within any organism, produces modifications of the electromagnetic field generated by said organism. Based on these principles, devices have been built for monitoring the heart, brain activity or to precisely determine an ovulation period. It was also Oschman who demonstrated that the muscle activity generates electromagnetic impulses that stimulate the cells regeneration, starting from the attraction of undifferentiated mesenchymal cells, and that any affection, such as a lesion caused by surgery, determines the modification of the magnetic field in the region of tissue trauma. In other words, the modification in the number of capillaries is accompanied by an increase of the magnetic “reluctance” of adjacent tissues. This increase is also determined by any local metal implants that disturb or prevent the normal, regenerating magnetic field resulting from the Schumman magnetic frequencies and the biological cell oscillations. From the aspects presented above, a conclusion may be drawn that the bio-integrability of an implant, impeded by the increase of the magnetic reluctance in the region due to wounding the tissues, due to tophus, microbes extant in the region as well as the insertion of any metal implants, can nevertheless be hastened by bringing the regenerating magnetic field to normal values.

Sisken and Walker have demonstrated that the frequencies of 2 Hz, 25 Hz and 50 Hz stimulate nervous regeneration, which is useful in implantology if hypoesthesia occurs following a surgical procedure. Sisken and Walker have also demonstrated that a 7 Hz frequency stimulates osseous regeneration and that a 10 Hz frequency stimulates ligament regeneration, which is for instance useful in parodontology for lowering teeth mobility. Sisken and Walker have also demonstrated that the 15 Hz, 20 Hz and 72 Hz frequencies stimulate the reformation of capillaries, this action being necessary after any surgical intervention involving osseous or soft tissue graft.

Herbert Frachlich demonstrated that an assembly of cells forming a tissue or organ has a specific frequency that regulates the physiology of said organ. If a big number of cells are affected, then the frequency cannot be emitted any longer and the disease or dysfunction occurs.

Those described above show that a healthy organ contributes to maintaining the health in its neighboring region, and a diseased organ cannot do this any longer and suitable steps should be taken to remedy the situation. When these measures must be applied in oral cavity in particular, attention must be in that the natural regenerating field is reduced, since the microbes and minerals extant in the tophus composition “steal” from the regenerating field intensity in order to mineralize themselves. It is known that the phenomena of healing by magnetic oscillations occur at low amplitudes of the field, with a magnetic induction of about 10⁻⁹ to 10⁻¹⁰ Tesla.

There are known dental kits comprising a device to generate an electromagnetic field to speed up the recovery of the tissue affected by the presence of an implant and its osteointegration, but also for the treatment of diseases, from a orromaxillofacial area such as periodontitis, which incorporate devices for generating a frequency electromagnetic field, preferably from 7, 692 Hz and electromagnetic radiation of 0.75 mT in the oral cavity, used in dental treatments, especially for bone and gingival cells overgrowth. The device consists of a circuit of electromagnetic field generation of extremely low frequency, and is connected with a local application device for producing of an electromagnetic field of extremely low frequency in the oral cavity, and a sinusoidal electric current, extremely low frequency, the circuit to produce electromagnetic field is composed of a quartz oscillator that generates a rectangular signal of initial frequency at high precision, which is successively divided through an integrated circuit whose output is obtained at a reduced frequency, from an integrated circuit type filter Butterworth, with rectangular signal is converted into sinusoidal signal, from a signal attenuator in eight steps, in order to provide a current in the area of 0.25 mT-2 mT every step of it, resulting in an increase of 0.25 mT of the induction magnetic field obtained between some polar parts of the device for the application local energy of the electromagnetic field, and from a constant current source; the device for the application of electromagnetic field in the oral cavity is a tweezers, made of permalloy with adjustable opening between its ends, ends which have, in their final part, some polar parts which apply to the area of interest, being placed in the middle of the tweezer a coil that generates the magnetic field of magnitude and shape imposed by the electromagnetic field generation of extremely low frequency. (patent No. RO 128805 B1 from Mar. 30, 2014)

The disadvantages of these kits consist of that the device for local application is relatively difficult supported by the patients, particularly those who have a high sensitivity and do not allow an application only on one, two or three teeth adjacent to each other, which increases the duration of treatment.

The use of low frequency is well known in the field of dentistry, to increase blood circulation to the gums, for example as in the international patent application No. WO 2006 001644. The device described in this application consists of a low frequency generator that is connected to a silicon electrode holder by a cable. Silicon electrode is applied to the gum in the area required for strengthening the blood circulation and to alleviate pain.

The main disadvantage of this solution is that technical, applying a magnetic field which should remain unmodified by applying a constant current, low frequency without variations from international application No. WO 2006 001644 cannot be applied to relatively long periods of time.

Another example of the electromagnetic field, ELF, comparable is presented in the application for patent no. CA 1202804, which describes the use of ELF for the correction the positional anomalies, of teeth. The effect obtained by this technique helps rebuild the soft tissues of the upper and lower jaw, through the application of permanent magnets, electromagnets or coils of electromagnetic induction that generates a field of very low frequency in the oral region. ELF frequency range is produced by movements of the mandible that interacts with some adjacent electrolytes in order to produce a regeneration stream.

A disadvantage of this technique is that the value of the current ELF obtained cannot be constant and cannot be adjusted according to the requirements of the treatment, whereas depends on human actions.

Application patent no. JP 2001 026529 presents a machine that is equipped with a low frequency generator and a high-frequency generator for cleaning Tartar or gum in order to stimulate lymphatic functions of gums and prevent and treat periodontal diseases.

The main disadvantage of this invention is that, again, contrary to the desired effects of the device and the method of this invention, low and high frequencies cannot be applied for long periods of time, and the device cleans only the teeth and can not be used for the purpose of gingival therapy.

Thus, devices known in the art generate very low frequency with amplitudes and intensities sometimes significantly below those attributable to terrestrial magnetism. However, such electromagnetic fields include a current component and displays oscillations of the same reason, so the effects at the cellular level of these appliances are not those expected in time and are supported relatively difficult by the patients, especially those who have a high sensitivity and do not allow an application only on one, two or three teeth adjacent to each other, which increases the duration of treatment

Previous research on the gingival cell cultures, whose results are presented in summary, in the international patent application No. WO 2012/093277 A1, showed that an electromagnetic field generation of extremely low frequency-ELF-and subjecting the organic cells to this field produces a significant regeneration effect on cells. Gingival cell cultures were placed in Petri dishes and were subjected to an electromagnetic field having various impulses and intensity for different time periods, when Petri dishes were placed within the Helmholtz-type structures.

The device used for the generation of electromagnetic field in this international application has two channels to generate electromagnetic pulses, each consisting of two oscillators with locking, each one generating a frequency ELF and functioning alternatively, so that only one oscillator from a channel operates at a specific time, according to a periodicity. In addition, the device also includes a final circuit and an induction coil that generates electromagnetic fields having the frequency of oscillator in the channel selected, combined with the frequency of an pilot oscillator selection and a circuit controlled by a pilot oscillator that alternates lock operation of oscillators, and change frequency emitted by each selected channel via two control signals. During operation of the appliance, in a manner disadvantageous, current did not remain constant and thus presents variations and oscillations within the same frequency range applied, wherein the applied magnetic field is disrupted as long as is applied on the cellular tissue.

For application in oral cavity of various healing techniques of treatment/treating, are used mouth guards, but at present there is no known a construction of a mouth guard used for the application of electromagnetic field in the oral cavity.

It is known a smart mouth guard presented in the international patent application No. WO 2014110548 A1 from Jul. 7, 2014, for diagnosis, quantification, and/or management, for example, bruxism. According to an example of realization, mouth guard tray includes a multitude of pressure sensors and processing circuit configured to supply the data from sensors to an external processing unit. Sensor assembly may also include temperature sensors, pH and/or motion sensors. The processor may be, for example, a smart phone or a computer.

The technical problem solved by the dental kit, in accordance with the invention, consists in ensuring a uniform electromagnetic field, simultaneously in several areas or on the entire length of the maxilla, and, implicitly, in reducing the duration of treatment, where the treatment can be continued in the absence of electromagnetic field generated in the oral cavity in which it is carried out or not an implant, and the patient may move during treatment, in the presence of the electromagnetic field.

The dental kit, withdraw the above mentioned disadvantages by that it comprises two external mouth guards, the first of which has three work-points, and the second one has two work-points, an external mouth guard with multiple work-points, a material consisting of a polymer with high polarity and a device generating an electromagnetic field which generates, inside the mouth guards, an uniform field with an induction field ranging between 0.7 . . . 0.9 mT, with a variation ranging between 5 . . . 10%, in the area subjected to the electromagnetic field, on a distance of 1 cm around the same point, and on a volume as high as possible in the areas where the tooth root or the implant is to be positioned, produced by a current of 200 mA, with a frequency ranging between 7 and 8 Hz, preferably 7.69 Hz, the field lines being perpendicular on the target tissue, which are placed in a box, and where

the mouth guard with three work -points consists of a curved body, that follows the anatomical shape of the jaw, comprising two walls curved exterior and respectively, curved frontal interior, bonding between them to some lower heads with a basic wall, the exterior wall having some guides, outside, lateral and respectively terminal and the interior wall having some other guides, inside, lateral and respectively frontal, said guides and the other guides being parallel to each other, two by two, so as to allow mounting in some spaces and in some other spaces delimited each other, the three active applicators, which generates an uniform electromagnetic field, on a distance of 1 cm around the same point, in the area located in front of their cellular tissue of the oral cavity, each applicator having in the composition a polar piece in the form of a rectangle, a core around that is placed, between a lid which has an ellipse-shaped and a plate, a coil, made from enameled copper wire with a diameter of 0.2-0.4 mm, in which

the external mouth guard, with two work-points consists of another curved body, that follows the anatomical shape of the jaw, comprising two walls curved exterior and respectively, curved interior, bonding them to some lower heads with a basic wall, the exterior wall having some guides outside, lateral, terminal, delimiting some elongated spaces, open at both ends, and the interior wall having some other guides inside, lateral, which delimiting some other spaces elongated open at both ends, which together with other spaces form two work-points in which are mounted two active applicators and in that

the external mouth guard that acts on the entire arcade consists of a metal curved body, that follows the anatomical shape of the jaw, comprising two walls parallel to each other, curved exterior and respectively, interior, bonded among themselves, upper or lower, depending on the use, on the upper or lower jaw of a superior wall, the body forming the metallic core of a coil made of copper wire, frontal, the coil having two electric conductors, frontal, through which it is carried out electrical connection with the device, on the outside, the coil being covered by a layer made of polyethylene or of the material; and in that the material is in the form of the sandwich being subsequently a body of a personalized internal mouth guard that follows the anatomical shape of the jaw; and in which

the device is a generator of sinusoidal low frequency current in 2 Hz-25 Hz range, with the level of output current 200 mA, maximum output voltage 24V, and is built around a microprocessor which generates the signals of low frequency current which generates an electromagnetic field of uniform induction intensity on a distance of 1 cm around the same point on a larger volume in areas that would be positioned dental root implant, of 0.75 mT-0.9 mT, and a direct digital synthesizer adapted to directly generate sinusoidal signal precisely with a variation from 2 to 50 Hz of high precision and stability led by the processor, the device containing a processing block, the synthesizer, a digital amplifier block with current reaction, an audio amplifier block, a clock block for real time, a block of memory, an on/off switch block and a power supply.

According to an aspect of invention, a body of the personalized mouth guard has some sockets placed in front of the teeth of the oral cavity, and if there are any missing teeth in their own front, between two sockets surrounding free space, is placed a blunt touching the gums.

According to an aspect of invention the personalized mouth, if it is made and fixed by gluing of one of the basic walls which belong to each of the mouth guards with three or two work-points, or where appropriate, of the wall of the mouth guard with multiple work-points.

According to an aspect of invention in the breaks between the use of trays another free personalized mouth guard can only be applied, in the oral cavity to cause more cells to migrate to the implant, leading to the recovery of the affected cells and keeping morphotype and phenotype of the cells that reach on the surface of the implant helping to better integrate in the socket bone.

According to an aspect of invention the material is in the form of a sandwich consisting of a polymer with high polarity, in which mineral granules are embedded with crystalline structure or substance or, preferably, natural, consisting of topaz, jade, calcium carbonate, octocalcic phosphate, silicates, quartz crystals, salt or natural ground, volcanic rocks, dust from animal horns or granules, zirconium oxide alone or in a mixture, the size of the particle being of 20 . . . 150 nm

According to an aspect of invention if it used the internal personalized mouth guard attached to one of the basic walls which belong to each of the guides with three or two work -points, the material that is carried out this mouth guard contains granules of zirconium oxide.

According to an aspect of invention the polar pieces are in contact with a lower wall of the basic wall, which in final is covered with a layer, made from a polymer with high polarity, preferably polyethylene with thick, preferably of 1 to 2 mm.

According to an aspect of invention said body is made with a 3D printer, preferably from polylactic acid and has between some exterior protuberances, that reinforce the guides, a length of 85.6 mm and the length of a profiled head of the basic wall and a profiled surface, outer the wall has preferably a value of 65.6 mm.

According to an aspect of invention said bodies are made of zirconium oxide.

According to an aspect of invention the coil is uniform wired, wire near wire, so as to be a uniform induction electromagnetic field in the outer edges of the wings parallel to each other, and when the current is raised to 400 mA, to determine in the continuance of the wings a volume where the field is uniform, in the depth of the bone.

According to an aspect of invention the mouth guard is made of two separate semi-mouth guards which together cover the entire jaw, each can be a semi-arch, each semi- mouth guard having frontal a coil, respectively, two frontal electric conductors, and respectively, through which it is carried out with the electrical connection with the device, thus, the electromagnetic field generated by each or both has a high uniformity, with a variance of less than 8%, on the whole area being treated.

According to an aspect of invention the processing block, consisting of the microprocessor, a display device and some connection jacks, generates through microprocessor the following signals:
clock signal for serial communication;
data signal for serial communication;
signal to pin nr 37, selecting the display device during communication;
signal selecting memory during communication with it;
signal selecting read-write memory;
signal selecting the write signal;
database signal for communication i2C;
clock signal for I2C communication;
signal switch of auto maintenance switch on (active on 1);
signal rectangle with variable fill factor for the generator for command output current level;
clock signal for synthesizer;
signal from flagging operation through the intermittent lighting up of the led;
read signal from memory and filtered from the generator out of pin 30 of the microprocessor;
signal coming out of the pin 1 of the microprocessor and to light backlight display device
input signals in the microprocessor:
signal measurement of output current that comes in the form of alternative and is numeric filtered in the microprocessor and measured;
signal of constant voltage divided from the supply voltage for battery monitoring;
signal given by the on/off switch to turn off the device
signal of the reference voltage for measurement current

According to an aspect of invention
the synthesizer contains a sinusoidal signal generator made with modulators which incorporates current reaction and limiting the signal

According to an aspect of invention
the digital amplifier block with current reaction, contains a linear amplifier of switching class D.

According to an aspect of invention
the audio amplifier block is an amplifier of low-voltage in power topology BTL of low voltage.

According to an aspect of invention
the memory block has an integrated circuit having a memory of 1024 Kbytes for uploading audio file and memory history of treatments and any information.

The dental kit, according to the invention, presents the following advantages:
ensuring a constant, uniform electromagnetic field, simultaneously in different areas of the jaw, or on the whole length of it reducing the duration of treatment;
allowing the continuation of the activation of the tissue at the cellular level in the orromaxillofacial area, while the mouth guard is not plugged into the device;
oral device is supported well during treatment of patients, without any effort on their part, being personalized;
relatively simple construction;
safety in operation.

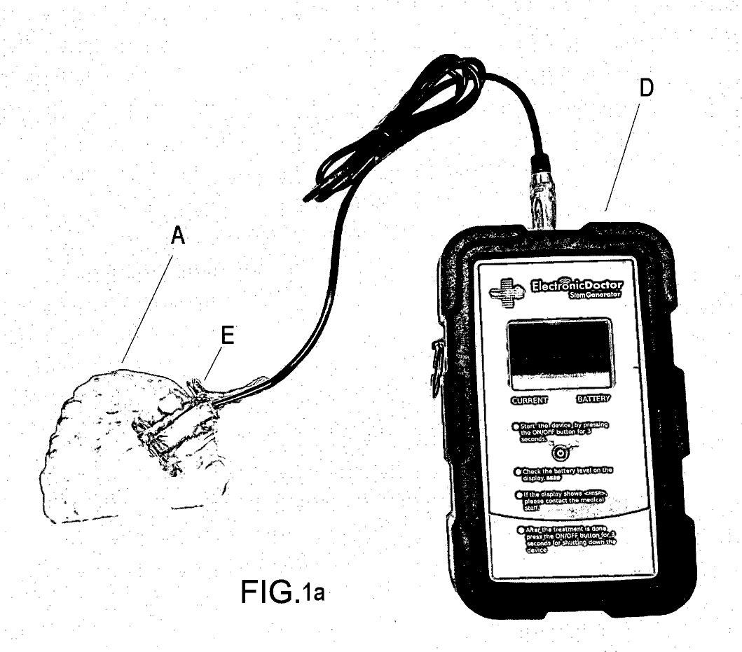

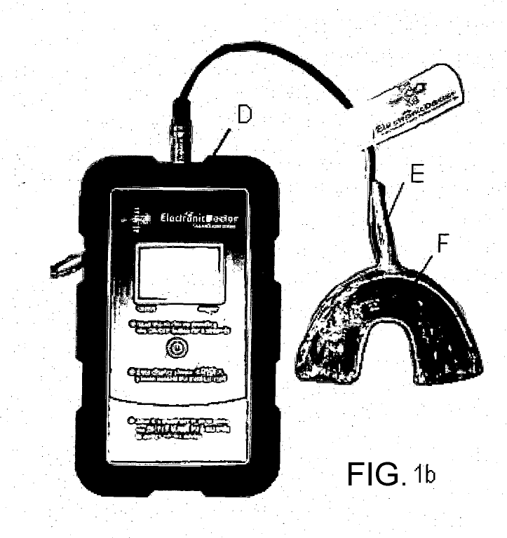

In the follows there are three versions of producing the dental kit, according to the invention, in relation with FIG. 1 . . . 39 that mean:

FIG. 1a, b—block diagram of dental kit, according to two examples of realization the invention;

FIG. 2, top view of a mouth guard with three work points, according to the invention;

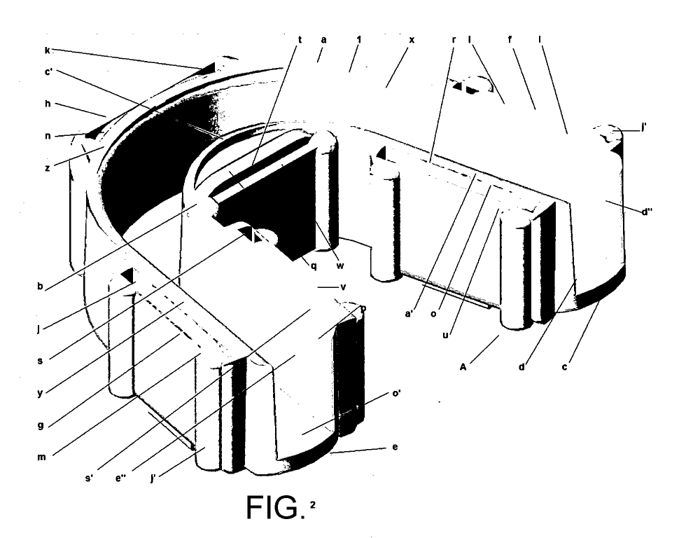

FIG. 3a—bottom view, in perspective, of a external mouth guard with three work-points, according to the invention

3b.—bottom view in perspective, of a external mouth guard with two work-points, according to the invention;

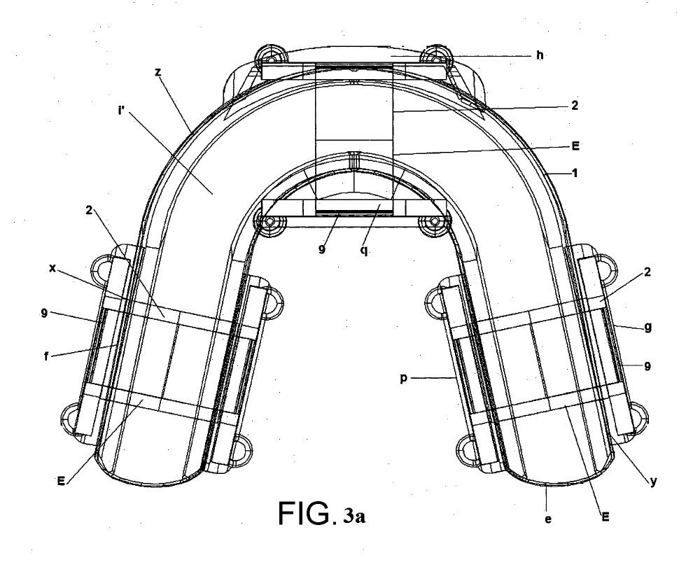

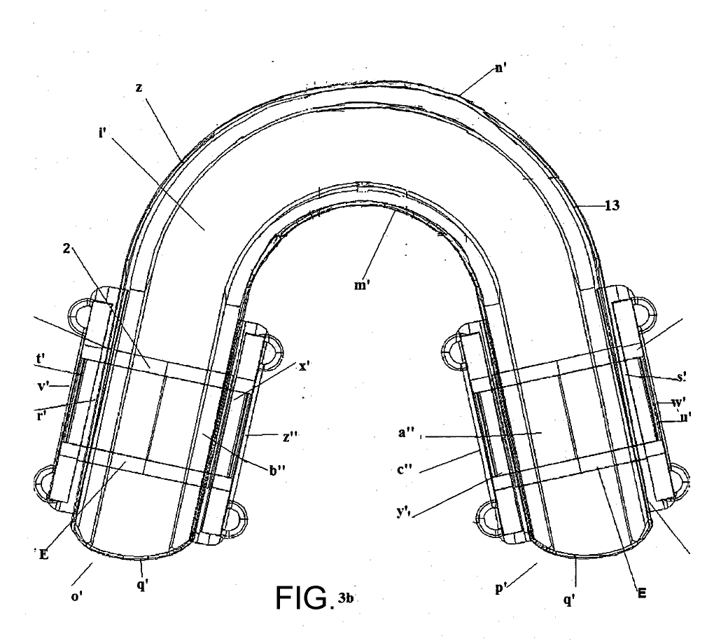

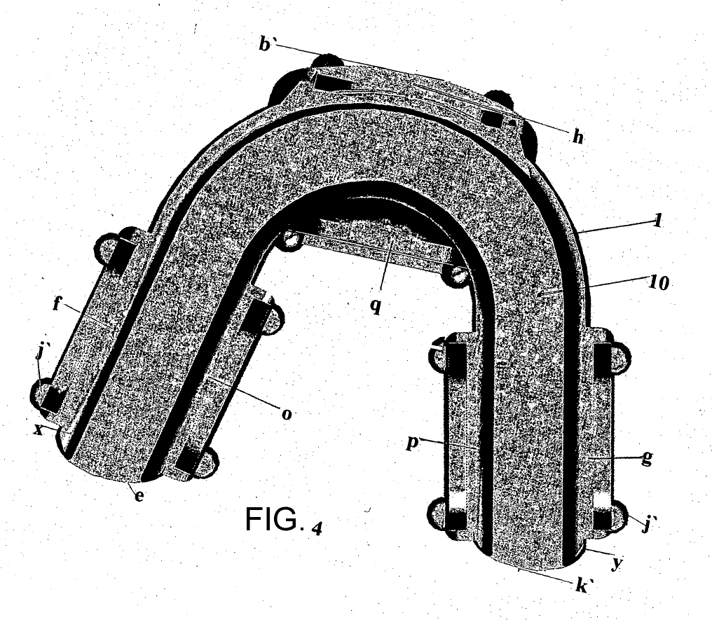

FIG. 4, bottom view of the coated mouth guard with a layer made of a polymer with great polarity;

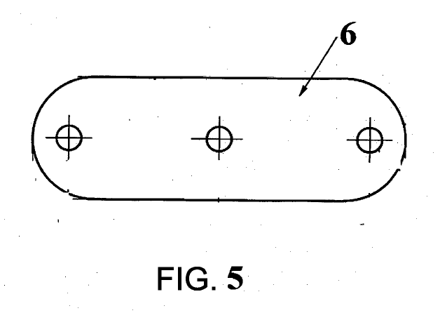

FIG. 5, top view of a core of active subassembly;

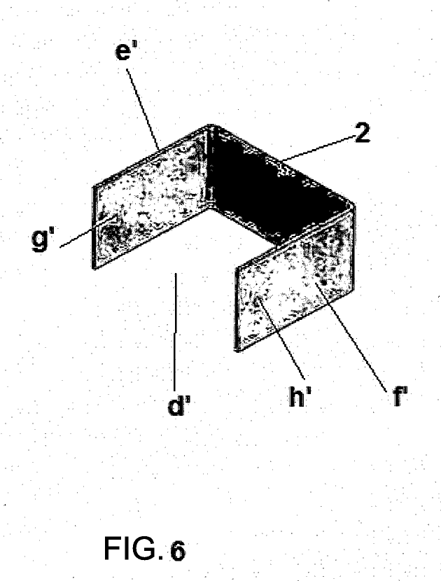

FIG. 6, view in perspective of the polar piece of active subassembly;

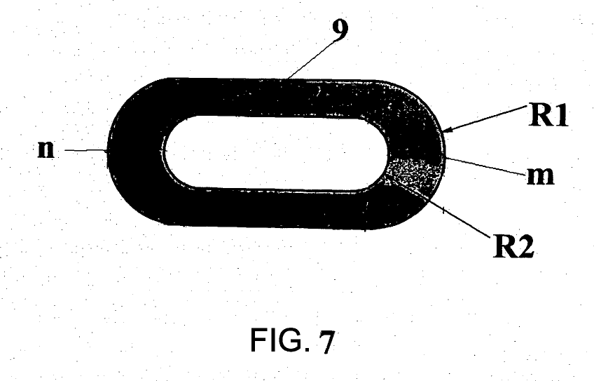

FIG. 7, view from the side of a coil of active subassembly;

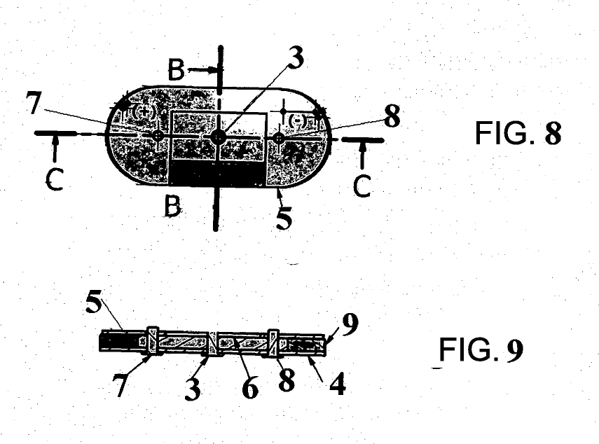

FIG. 8, view from the side of the lid of the coil;

FIG. 9, section after the A-A plane, rendered in FIG. 7 through active subassembly;

FIG. 10, section after a longitudinal plane through the polar piece that the polar coils are fixed

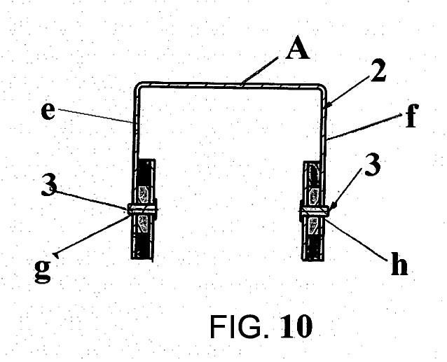

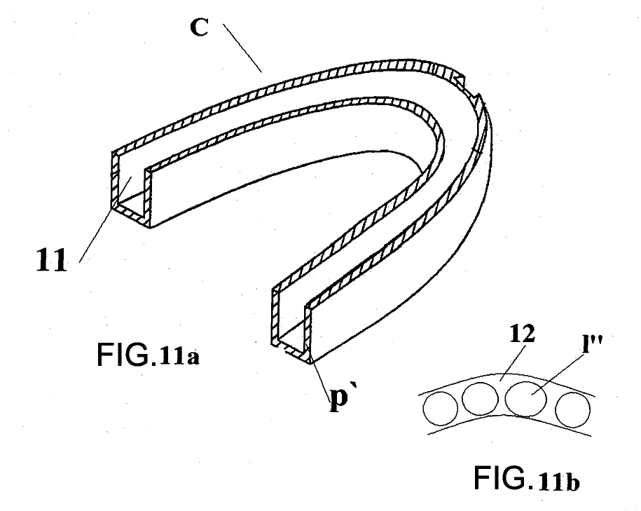

FIG. 11a—top view perspective of a internal mouth guard;

11b—view of material for internal mouth guard;

FIG. 12, top view of mouth guard with multiple work-points;

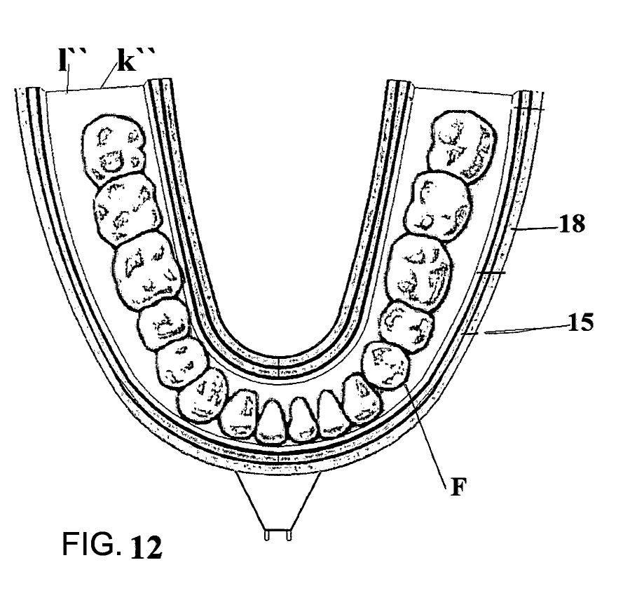

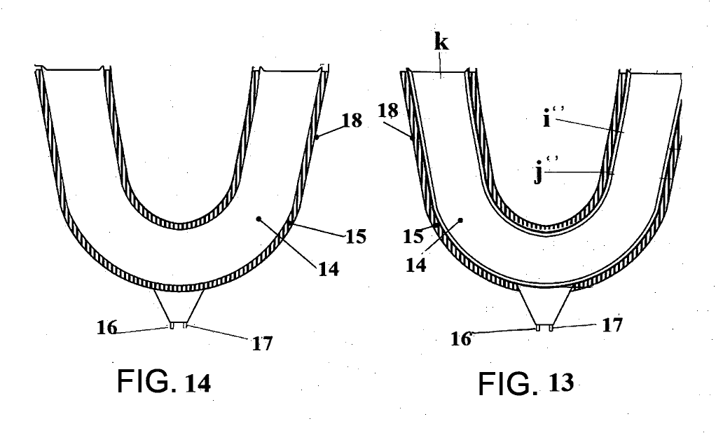

FIG. 13, top view from a mouth guard with multiple work-points, in another embodiment;

FIG. 14,—top view from a mouth guard with multiple work points in another embodiment;

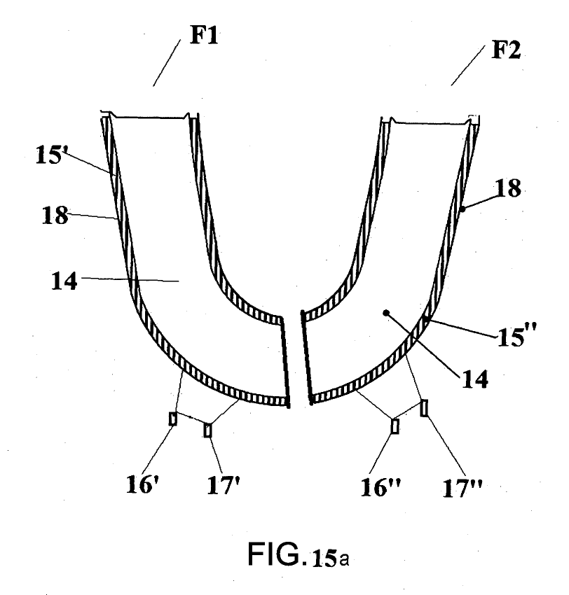

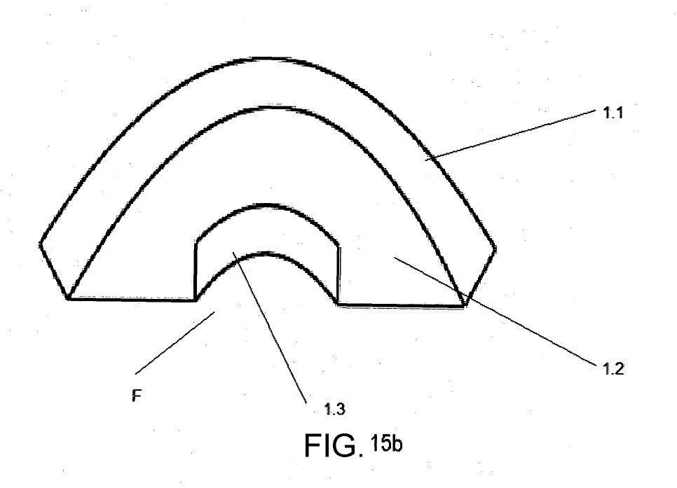

FIG. 15a,—mouth guard made of two semi mouth guards, a further example of realization;

FIG. 15b, mouth guard made up of three sections, according to another example of realization

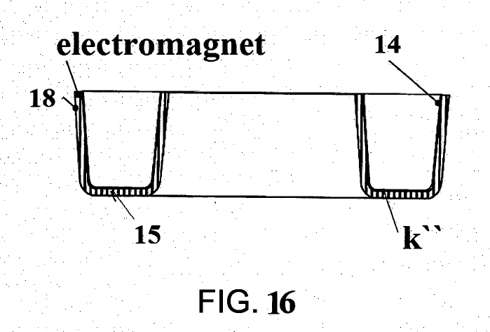

FIG. 16,—view at the rear of mouth guard the sight from FIG. 13;

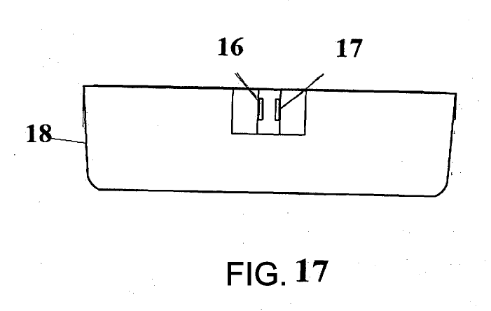

FIG. 17,—view of the front sight presented in FIG. 14;

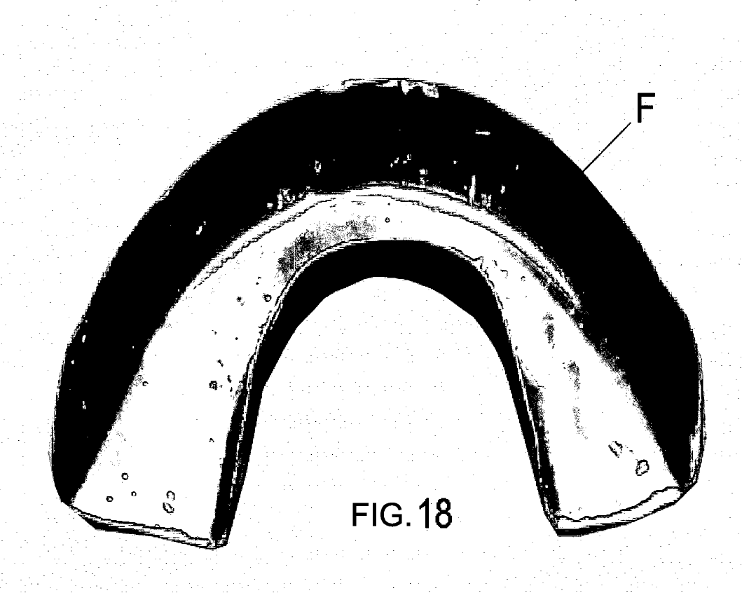

FIG. 18,—view of internal mouth guard;

FIG. 19,—view in perspective of an internal mouth guard according to another example of realization;

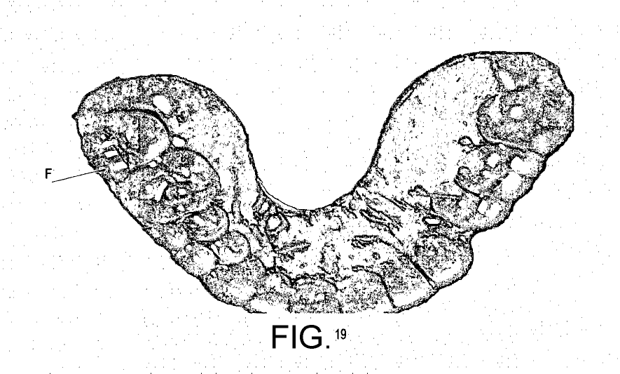

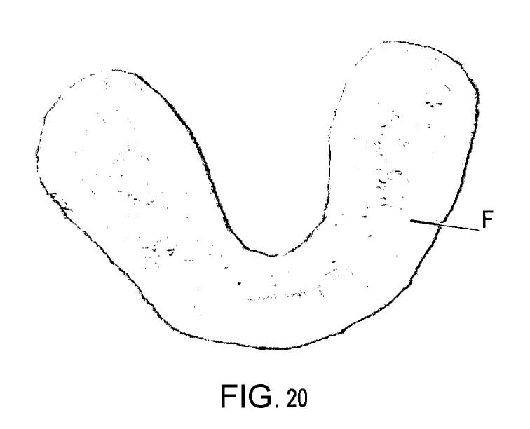

FIG. 20,—view in perspective of an internal mouth guard, according to another example of realization;

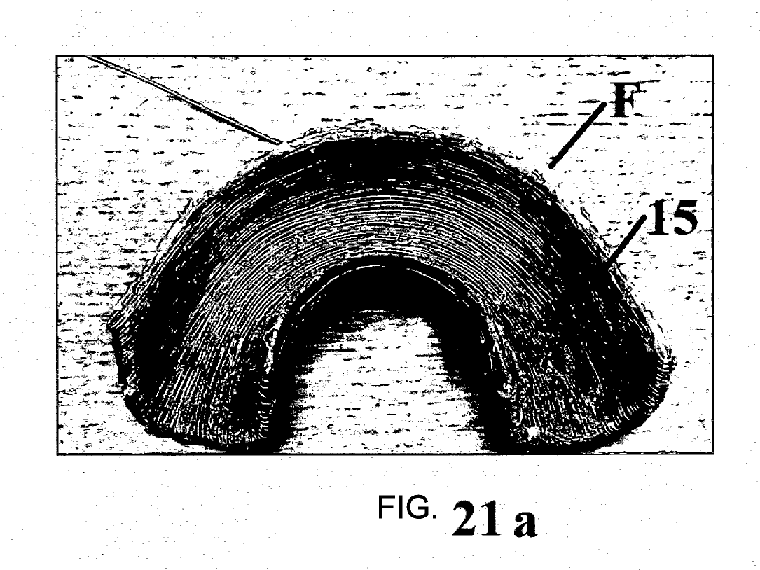

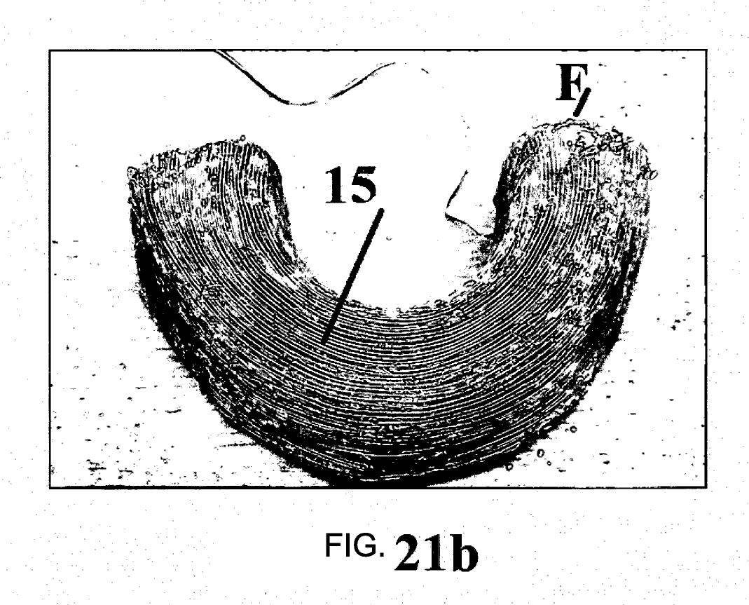

FIG. 21—a, b—view in perspective of an internal mouth guard, according to another example of realization;

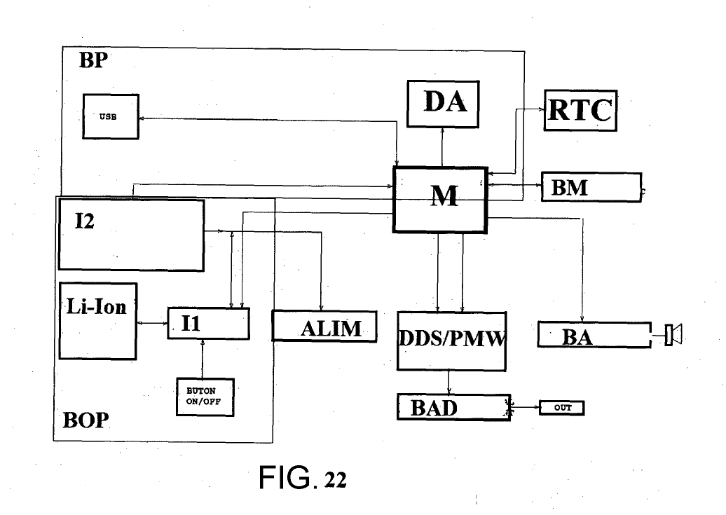

FIG. 22:—block diagram of the device for generating electromagnetic field according to an example of realization of dental kit;

FIG. 23,—detailed electronic scheme of the device for generating electromagnetic field according to an example of realization of dental kit;

FIG. 24,—electronic scheme of processing device for generating electromagnetic field according to an example of realization of dental kit;

FIG. 25,—electronic scheme of sinusoidal generator device for generating electromagnetic field according to an example of realization of dental kit;

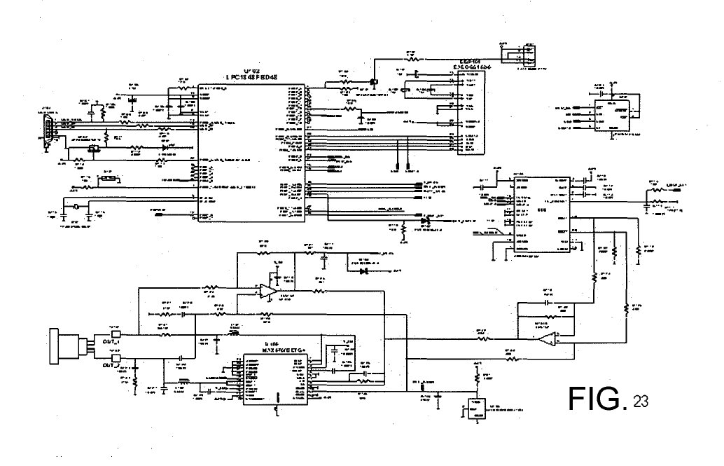

FIG. 26,—electronic scheme of amplifier digital electromagnetic field generation device, according to an example of realization of dental kit;

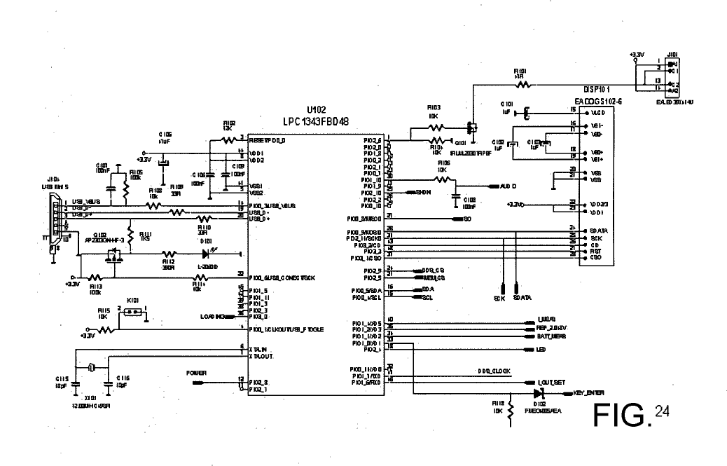

FIG. 27,—electronic scheme of audio amplification device for generating electromagnetic field according to an example of realization of dental kit;

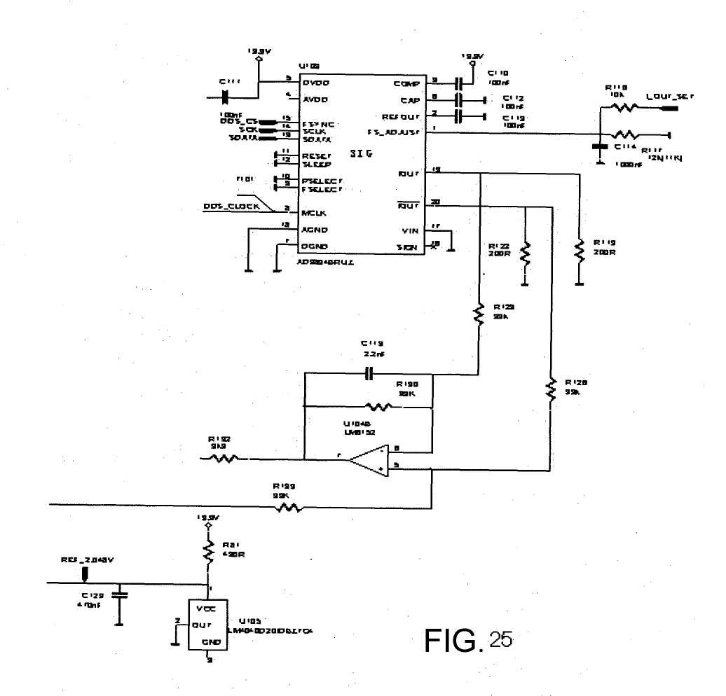

FIG. 28,—electronic scheme of real-time clock block of the device for generating electromagnetic field according to an example of realization of dental kit;

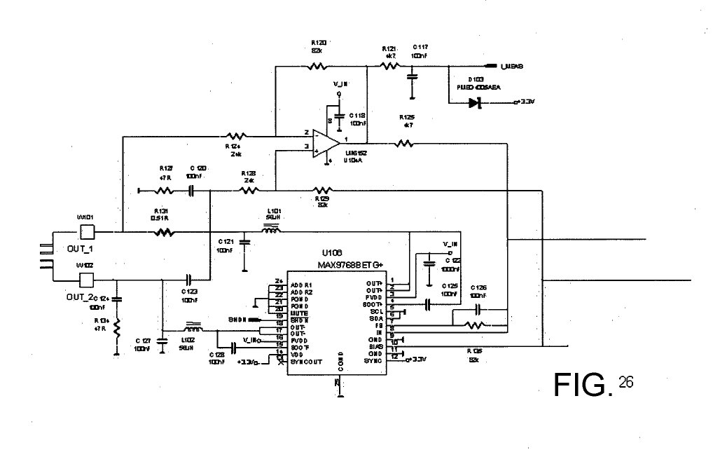

FIG. 29,—electronic scheme of block diagram memory generating electromagnetic field according to an example of realization of dental kit;

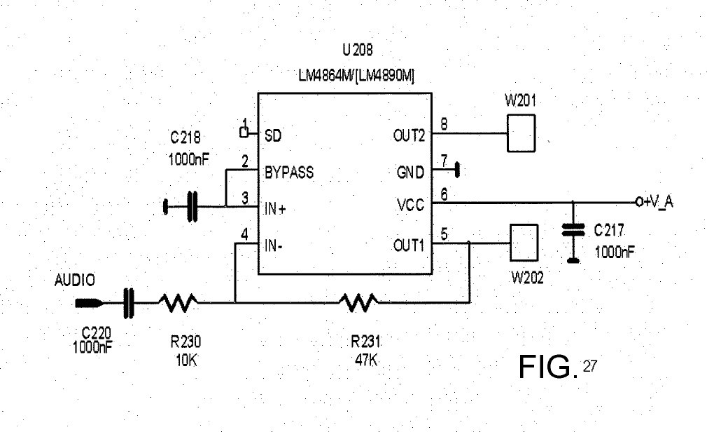

FIG. 30,—electronic scheme of diagram start/stop block device for generating electromagnetic field according to an example of realization of dental kit;

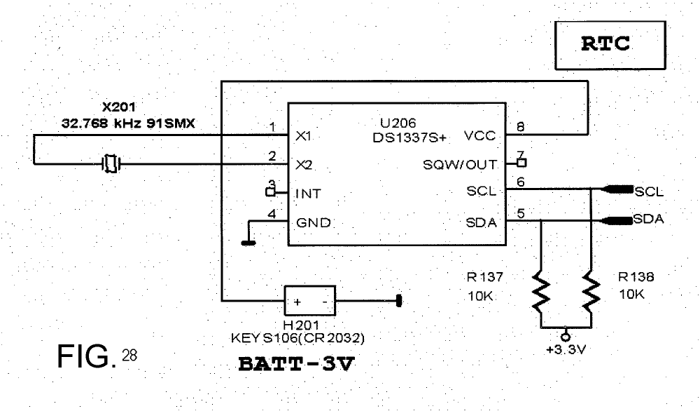

FIG. 31,—electronic scheme of power device for generating electromagnetic field according to an example of realization of dental kit;

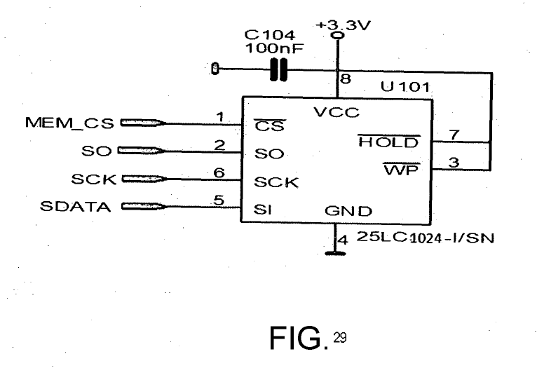

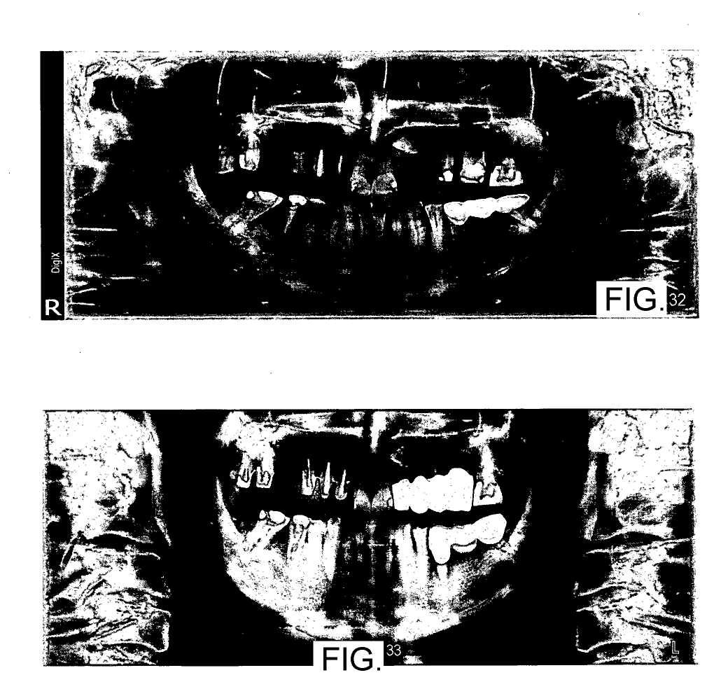

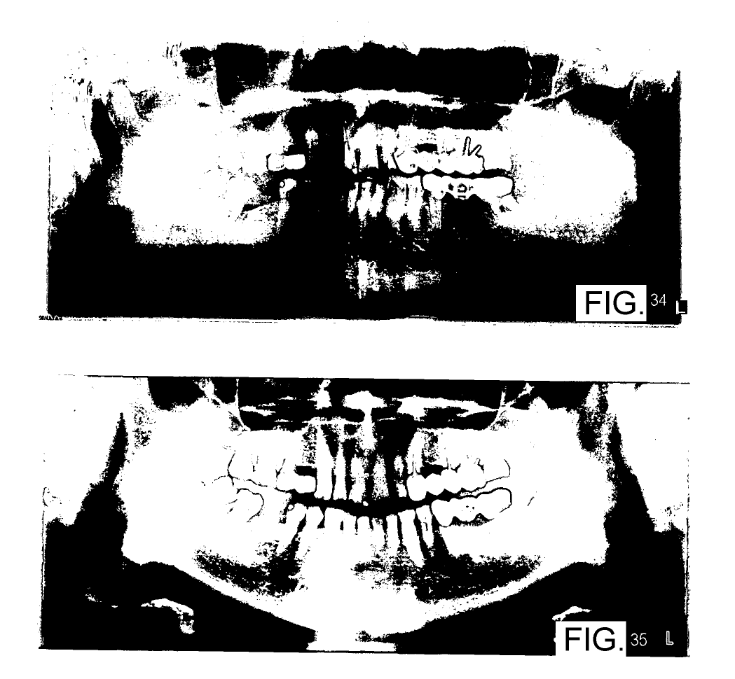

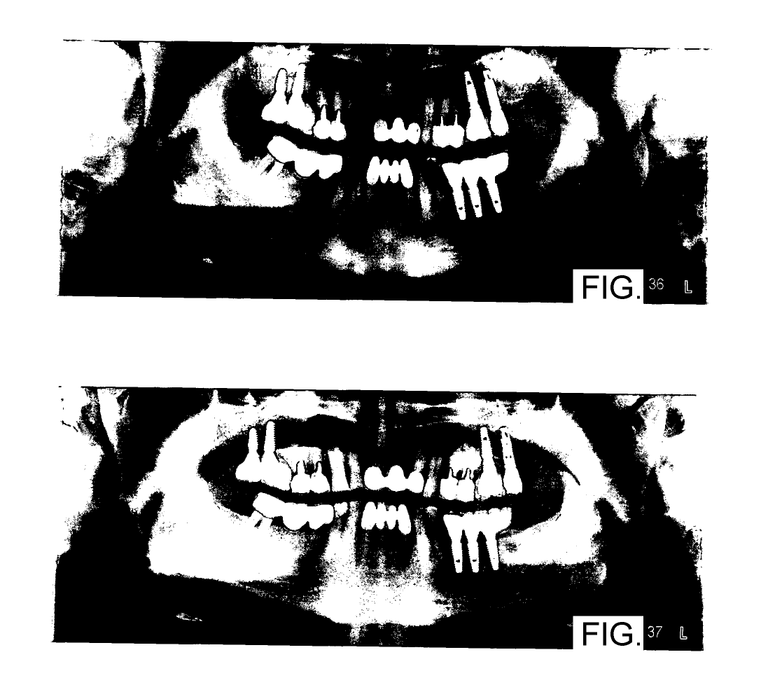

FIGS. 32-39—radiological images made on patients before and after treatment using dental Kit, according to the invention.

Dental kit, according to the invention comprises an external mouth guard A which has three work-points, an external mouth guard B with two work-points, an external mouth guard F with multiple work-points, a material 12 consisting of a polymer with high polarity and a device D generating an electromagnetic field placed in a box (not figurate). The assembly consisting of the device D and the applicators E is made in such a way to generate, inside the mouth guards A or B or F, an uniform field with an induction field ranging between 0.7 . . . 0.9 mT, with a variation ranging between 5 . . . 10%, in the area subjected to the electromagnetic field, on a distance of 1 cm around the same point, and on a volume as high as possible in the areas where the tooth root or the implant is to be positioned, produced by a current of 200 mA, with a frequency ranging between 7 and 8 Hz, preferably 7.69 Hz, the field lines being perpendicular on the target-tissue.

The mouth guard A consists of a curved body 1, that follows the anatomical shape of the jaw, comprising two walls a and b curved exterior and respectively, curved frontal interior, bonding between them to some lower heads c and d with a basic wall e.

The exterior wall a has some guides f, g and h, outside, lateral and respectively terminal that delimitate some elongated spaces i, j and k, open at both sides. Some short exterior walls l, m and n of the guides f, g and h are inner planed.

The interior wall b has some other guides o, p, and q, inside, lateral and respectively frontal, delimiting some elongated spaces r, s and t open at both ends that together with the spaces i, j and k form three work-points. Some exterior short walls u, v and w of the guides o, p and q are inner planed.

The curved a and b are equal distanced between them.

The guides f, g and h and the other guides o, p, and q are parallel to each other, two by two, so as to allow mounting in some spaces i, j and k and in some other spaces r, s and t delimited each other, the three active applicators E, which generate an uniform electromagnetic field, on a distance of 1 cm around the same point, in the area located in front of their cellular tissue of the oral cavity. Each applicator E has in the composition a polar piece 2 in the form of a rectangle with an open d′ on one side, provided some arms e′ and f′, parallel to each other, with one of the penetrated low holes g′ and h′.

From arms e′ and f′ in the front of the g′ and h′ are fixed using a rivet 3, a front plate 4 and a lid 5, between them is placed a core 6 that is penetrated by the rivet 3.

Initially, the lid 5, the core 6 and the plate 4 are joined together among themselves by means of rivets 7 and 8, then a lid 5, a coil 6 and a plate 4 are joined together by each of the arms e′ and f′ with a rivet 3.

Around each core 6 is placed between the lid 5 that preferably has an ellipse shape, and plate 4, between which is a distance, preferably of 2.5 cm, a coil 9, made from enameled copper wire with a diameter of 0.2-0.4 mm. The ends of coil 9 are pierced through some penetrated holes not figurate, practiced in the lid 5 which is achieved preferably from glass-textolite, being tinned of it.

The lid 5 has the form of an ellipse and preferably has a length of 27.0 mm, height 12.0 mm, and a thickness of 0.5 mm, and the distance between centers of pierced holes crossed the rivets 7 and 8 is preferably 14.5 mm.

In the front of the guides f, g and h of the exterior wall a and respectively in front of the guides o, p and q of the interior wall b in the spaces i, j and k, and respectively in the spaces r, s and p are thus placed the three applicators E, such that the polar pieces 2 are in contact with a lower wall i′ of the basic wall e that in final is covered with a layer 10, made from a polymer with high polarity, preferably polyethylene with thick of 1 to 2 mm.

The polar piece 2 is carried out, preferably from steel and can have one of the lengths of 55 mm, 58 mm or 61 mm and a width of 11.4 mm. Depending on your needs, the polar piece 2 may have one of the odds of opening d′ of 19 mm, 22 mm, 25 mm or 28 mm, resulting after fixing of the arms e′ and f′ of the coil 9 the use odds 15 mm, 18 mm, 21 mm or 24 mm.

The body 1 is made with a 3D printer, preferably from polylactic acid and has between some exterior protuberances j′, that reinforce the guides f and g, a length of 85.6 mm and the length of a profiled head k′ of the basic wall e and a profiled surface l′, outer the wall n has preferably a value of 65.6 mm.

The core 6 has the shape of an ellipse, and preferably has a length of 18 mm, 6.0 mm in height, and has three pierced, holes not plotted in the figures, which are penetrated of rivets 8, 3 and 7, the distance between the centers of the holes is 7.25 mm and the thickness is 1.5 mm.

A coil 8 has an ellipsoid shape, preferably some vaulted heads m′ and n′ with some outer curvature rays R₁ and respectively R₂, and, with a total length of 27.0 mm, height 12.0 mm, distance between the centers of the ellipse of 14.5 mm, and a thickness of 1.5 mm.

In a space o′ bounded by the walls a and b can be positioned and glued in a way itself known, as for example with acrylic material, an internal personalized mouth guard C made by the dentist function of the anatomical particularities of the patient, to be worn by the patient according to the prescriptions of the dentist. The personalized mouth guard C has a body 11 which seeks the shape and contour of the upper and respectively the lower jaw, made of material 12, consisting of a polymer with high polarity, in which mineral granules are embedded with crystalline structure or substance or, preferably, natural, consisting of topaz, jade, calcium carbonate, octocalcic phosphate, silicates, quartz crystals, salt or natural ground, volcanic rocks, dust from animal horns or granules, zirconium oxide alone or in a mixture, which are designed to decrease the resonance and local reluctance for environmentally sound frequency magnetic cellular adjacent teeth and/or implant with a value of intensity 7.69 Hz, or induction of uniform in the area at least at the same point, the particle size being of 20 . . . 150 nm.

In order to obtain the material 12 from which it is made the body 11 after mixing, preferably granules, zirconium oxide or zirconium oxide and quartz with polymer with high polarity at the melting temperature of his that can be obtained is polyethylene mixture melted and then cooled and regranulated, and these grains are pressed from hot or cold in a mold to obtain the desired shape. Further these granules are hot pressed between two or more polymer foils polar, obtaining a material 12 sandwich type so that no granules from chosen materials coming into direct contact with tissues, thus reducing the risks of adverse effects from using the mouth guard C together with the mouth guards A or B.

The body 1 can be achieved the best manner of zirconium oxide, through processing, having the walls a and b fitted with guides f, g and respectively h, and with guides o, p and q where are mounted three subassembly E.

In this constructive embodiment the body 11 of the personalized mouth guard C is made from a material 12 in the form of sandwich made of polyethylene which is embedded in zirconium oxide powder.

Thus it is ensured a more pronounced decrease of local reluctance and a subsequent greater resonance of magnetic field, where the implant is made of titanium and zirconium oxide or zirconium oxide and, preferably, in the form of powder or granules pressed from the minerals or substances with crystalline structure. By using the mouth guard C in this case, is obtained a better stabilization during treatment, no matter if the patient moves or has a tendency to move during treatment.

During using the dental kit, in accordance with the invention in the oral cavity, where was performed at least one dental implant that has constructive components made from titanium and/or only zirconium oxide, one by one, in each area located in front of the implant after placing the body 1 and the mouth guard C in oral cavity, covering appropriate upper or lower gum, is carried out the link between the ends of coil 8 with device D.

In the breaks between the use of mouth guard A or B or F another free personalized mouth guard C can only be applied, in the oral cavity to cause more cells to migrate to the implant, leading to the recovery of the affected cells and keeping morphotype and phenotype of the cells that reach on the surface of the implant helping to better integrate in the socket bone.

These mineral granules or crystalline structure substances of the composition material 12 generate low frequency oscillations of resonance with electromagnetic waves biocompatible of space.

The mouth guard B consists of a curved 13 body, that follows anatomical shape the jaw, comprising two curved walls n′ and m′ exterior respectively, interior, bonded among themselves at some lower heads o′ and p′ with a basic wall′ q.

The outside wall n′ has some guides r′ and s′ outside, lateral, terminal, delineating some elongated spaces t′ and u′ open at both ends. The guides r′ and s′ with some short exterior walls v′ and w′ are planed inside..

The interior wall m′ has some other guides x′ and y′, inside, lateral, that delimitate some other spaces z″ and a″ elongated open at either end that together with spaces t′ and u′ formed two work-points. The guides x′ and y′ have some short exterior walls b″ and c″ planed inside.

The dimensions of the body 13 are identical to those of the body 1.

The guides r′ and s′ and guides x′ and y′ are parallel to each other two by two, so as to allow the mounting in the corresponding spaces t′ and z′ and respectively u′ and a″ of an active applicator E.

The body 13 can be achieved with a 3D printer, preferably from polylactic acid or mostly from zirconium oxide, through processing. In this latest variation constructive, the walls m′ and ‘n’ are provided with guides s′ and respectively, r′ with guides x′ and y′ in which are mounted two applicators E.

After making the personalized mouth guard C, it is glued with a material such as acrylic wall of the basic wall q′ into a space h′ delimited of the spaces m″ and n″.

The mouth guard F consists of a metallic curved body 14, that follows the anatomical shape of the jaw, comprising two curved walls i″ and j″, exterior respectively, interior, unite among themselves, upper or lower, depending on the use, on the lower or upper jaw by a basic wall k″. The body 14 constitutes the core of a metallic or paramagnetic coils 15 made preferably from a copper wire. Preferably copper wire has a diameter of 0.2-0.4 mm, and it uses a number of wires of 140-150.

Frontal, the coil 15 has two electric frontal conductors, 16 and 17, through which it is carried out the electrical connection with the device D. On the outside, the coil 15 is covered by a layer 18 made of polyethylene or the material 12.

Therefore, the treatment of bio reactivation with electro-magnetic field of very low frequency emitted by the device (D), shall be made through the coil 9 or 15 disposed in the applicator (E).

Electromagnetic field applicator (E) (CEM) has a U-shape, including between the two poles 2 the area that require regeneration. The distance between the poles is variable depending on the thickness of the arch in the area of application, ranging between 15 and 21 mm. The depth from which we have the uniformity of CEM is the maximum length of the applicator. The applicator E in turn is placed and immobilized on the interior mouth guard A or B for each patient, in the area requiring treatment. In this way is certainty that the applicator E will act strictly over the desired area without being able to be mobilized by the patient.

In order to accomplish the mouth guards A or B of supporting the applicators E, are needed the fingerprinting of the maxillary arches, casting the work models from gypsum and making the mouth guards A or B from thermoplastic foil of 2 mm thick. The applicators E are bonded and secured on the area that requires treatment and connected through a cable to the device D transmitter of CEM. On an arch can be installed only two (mouth guard B) or three (mouth guard A) such applicators E simultaneously due to very limited space of the oral cavity, including between their poles a segment of maximum 3 teeth. The patient is instructed practical about the way to apply the mouth guard A or B or F on the dental field, how hygienic, how you have connected the kit and the number of exposure to CEM.

The mouth guard F may be or not covered of material 12, or before starting the use of the mouth guard F, can be carried out by the dentist the personalized mouth guard C of the patient from material 12, after which the mouth guard C is placed in a space l′ delimited laterally by foil 18 next to the walls i″ and j″ or are mounted of bands 19 and 20 and then it is bonded by sticking from the wall k″ The coil 15 is wired in addition, wire near wire, such there is a uniform magnetic field induction, with an uniform value in the outer edges of the wings parallel to each other.

In another embodiment, the mouth guard F is made of two separate semi-mouth guards F1 and F2 which together cover the entire jaw, each can be a semi-arch, each semi- mouth guard F1 and F2 having frontal a coil 15′, respectively 15″, two frontal electric conductors, 16′ and 17′ and respectively 16″ and 17″, through which it is carried out with the electrical connection with the device D, thus, the electromagnetic field generated by each F1 or F2 or both F1 and F2 has a high uniformity, with a variance of less than 8%, on the whole area being treated.

In another embodiment, the mouth guard F consists of three sections, F1, F2, F3 sticking together such to have a dental arch shape. The material is made the support for wiring is a tinplate.

The three sections of F1, F2, F3 of the mouth guard F thus made are

1) outer wall 1.1 (vestibular)

2) Base 1.2

3) inner wall 1.3 (lingual)

The walls are welded on the base so as to form a right angle

The thickness of the outer wall 1.1 is 1 mm and variable height (13 mm, 14 mm, 15 mm)

The base 1.2 (the receiver area) is 0.40 mm thickness, variable width and length, so that it can encompass in receiver area most dimensions of dental arches (22 mm with 65 mm)—small (25 mm with 70 mm)—medium, (27 mm with 75 mm)—large

The inner wall 1.3 has the thickness of 0.40 mm and variable height (13 mm, 14 mm, 15 mm).

Combining these values is made a series of 9 individual mouth guard models with the following dimensions: R=Receiver LT=lengths of the walls.

Entire sheet metal bracket must be with a band of silicone paper and applied with a special gun.

Windings is made by wrapping wire of copper Ø0 40 mm running the following route

For a mouth guard F of large size, winding starts on the outer side of the vestibular wall from the center of it from the free side and continues on the outside to right, continuing on the inner side of the wall and continuing on the outer side of the vestibular wall until it reaches immediately below the initial starting place and then continue the wrapping process {about 25 for (H15 mm) about 23 for (H14 mm), about 20 for (H13 mm)}. When you reach with the previously presented last wire at the base of the outer wall of the receiver area starts winding of the base (receiver area) with 33-35 wires that continues with winding on outside base 1.2, until is winding up in the receiving area at the base of the wall 1.3 . After is finished winding in the receiving area of the base 1.2 switch to winding the inner wall (lingual). It is winding the inner wall 1.3 with {about 25 wires for (H15 mm), about 23 wires for (H14 mm), about 20 wires for (H13 mm)}

This procedure should be repeated with another layer of windings for the entire mouth guard F (two walls 1.1, 1.3 and the base 1.2) continuously winding wire near wire as uniformly as is possible especially in the receiving area where the magnetic field must have minimum non-uniformity (under 10%)

It reached winding of the top of the outer wall 1.1 from where it began winding and proceed with the third layer of the only of the outer wall 1.1 {(about 25 wires for (H15 mm), about 23 wires for (H14 mm), about 20 wires for (H13 mm)} placed as uniformly as is possible.

For a mouth guard F of medium size (R is 25 mm, LT is 70 mm, H is 13 mm, 14 mm, 15 mm), winding starts on the outer side of the vestibular wall from its center from the free side and continues on the outside to right, continuing on the inner side of the wall and continuing on the outer side of the vestibular wall until it reaches immediately below the initial starting place and then continue the wrapping process {about 25 for (H15 mm) about 23 for (H14 mm), about 20 for (H13 mm)}.

When you reach with the previously presented last wire at the base of the outer wall of the receiver area it starts the winding the base (receiver area) with 33-35 wires that continues with winding on outside base 1.2, until is winding up in the receiving area at the base of the wall 1.3.

After is finished winding in the receiving area of the base switch to winding the inner wall 1.3 (lingual). It is winding the inner wall 1.3 with {about 25 wires for (H15 mm), about 23 wires for (H14 mm), about 20 wires for (H13 mm)} in receiver area that continued on the outer area of the inner wall 1.3.

This procedure should be repeated with another layer of windings for the entire mouth guard F (two walls and the base) continuously winding wire near wire as uniformly as is possible especially in the receiving area where the magnetic field must have minimum non-uniformity (under 10%)

It reached winding of the top of the outer wall 1.1 where it began winding and proceed with the third layer only of the outer wall 1.1 {(about 25 wires for (H15 mm), about 23 wires for (H14 mm), about 20 wires for (H13 mm)} placed as uniformly as is possible.

For a mouth guard F of small size, (R is 22 mm, LT is 65 mm, H is 13 mm, 14 mm, 15 mm) winding starts on the outer side of the wall from the center of the vestibular wall of free side and continues on the outside to right, continuing on the inner side of the wall and continuing on the outer side of the vestibular wall until it reaches immediately below the initial starting place and then continue the wrapping process {about 25 for (H15 mm) about 23 for (H14 mm), about 20 for (H13 mm)}. When you reach with the previously presented last wire at the base of the outer wall 1.1 of the receiver area it starts winding the base 1.2 (receiver area) with 33-35 wires that continues with winding on outside base 1.1, until is winding up in the receiving area at the base of the inner wall. After is finished winding in the receiving area of the base 1.2 switch to winding the inner wall 1.3 (lingual). It is winded the inner wall 1.3 with {about 25 wires for (H15 mm), about 23 wires for (H14 mm), about 20 wires for (H13 mm)}

This procedure should be repeated with another layer of windings for the entire mouth guard F (two walls and the base) continuously winding wire near wire as uniformly as is possible especially in the receiving area where the magnetic field must have minimum non-uniformity (under 10%)

It reached winding of the top of the outer wall where it began winding and proceed with the third layer of the only outer wall 1.1 {(about 25 wires for (H15 mm), about 23 wires for (H14 mm), about 20 wires for (H13 mm)} placed as uniformly as is possible.

When were winding all three layers of wire on the outer wall 1.1 it will leave the final wire near the free edge where it was begun, and after to make electrical measurements with tesla meter to add wires if needed. When the sample measurements with tesla meter is satisfactory is finished metal wired base.

Electrical connection with textolite plate (connector) will be made at the center of the outer wall vestibular wall orientated as much towards the area of the applicator base E for the embarrassment does not occur of the patient's lip during use.

At the end of the winding F it can be concluded that it has 3 layers of wires on the external wall, two the base and two on the inner wall.

Once it is established (metrics) that the mouth guard is in optimum operating parameters it is cabling with an electrical cord fitted with a medical plug and is sent the base metal wiring in dental technical laboratory to be in foiled at a special vacuum forming device and UV-sterilized.

Apart from this mouth guard C fixed in the mouth guard F dentist can make a personalized mouth guard C form material 12 which were to be worn by the patient, in accordance with the prescriptions of the dentist.

The personalized mouth guard C is made of body 11 which has sockets l″ placed in the front of the teeth of the oral cavity, and if there are any missing teeth, between two sockets l″ surrounding free space is preferred to be placed a blunt m″ touching the gums.

The device D for the application of the electromagnetic field is the generation of electromagnetic field of a very low frequency, and is thus built as to generate inside of the mouth guards A or B or F a uniform field of induction comprised in the range between 0.7-0.9 mT, with a variation between 5-10% in the area subjected to the electromagnetic field, a distance of 1 cm around the same point on a larger volume in areas that would be positioned dental root or implant, produced by a current of 200 mA, ranging between 7 and 8 Hz, preferably 7.69 Hz

In order to obtain the above parameters, according to an example of realization, the device D is a generator of sinusoidal low frequency current in 2Hz-25Hz range, output level of 200 mA, maximum output voltage of 24 V RMS. Power is achieved through a battery of Li-ion accumulators.

According to an example of realization, the device D is built around a microprocessor M that generates current signals of low frequency.

With the device D, the field frequency ELF is only one and is already fixed, and its uniform induction in the area/target areas is substantially 0.75 mT . . . 0.9 mT, so, as you may be somewhat greater at the mouth guards level, will be potentially up to 3 mT, if you use the current of 400 mA for the depth of the bone.

Normally, for generating sinusoidal signal would have several possibilities:

a. Using a high-frequency generator and programmable frequency dividers for frequency in the form of rectangular signal and then filtered to get sinusoidal shape. This generates some signals with amplitude depends on the tolerance of the components in filter and discrete resolution due to the divider. This variant is not satisfactory.

b. Building a sinusoidal oscillator directly at work frequency but it wouldn't have the precision and stability.

c. Using direct digital synthesis variant, variant that is preferred and will be detailed below.

Thus, in order to obtain a continuous signal constantly and sinusoidal of extremely low frequency, according to one example of realization of circuits of the device D, it contains a Digital Synthesizer of constant current DDS adapted to generate directly sinusoidal signal precisely with a variation from 2 to 50 Hz, which is set at a frequency of between 7 and 8 Hz preferably 7.69 Hz Signal generated by DDS has a high accuracy and stability led by processor M.

According to an example of realization, D contains a processing block BP, the synthesizer DDS containing a block generator sinus PMW, a digital amplifier block BAD with current reaction, an audio block amplifier BA, a clock block RTC for real time, a block of memory BM, an on/off switch block BOP and a power unit ALIM.

The processing block BP, consisting of the microprocessor M, a display device DA and some connection jacks USB. The microprocessor M, generates the following signals:
clock signal SCK for serial communication;
data signal SDATA for serial communication;
signal CSD to pin nr 37, selecting the display device DA during communication;
signal MEM CIP SELECT selecting memory during communication with it;
signal SO selecting read-write memory;
signal DDS_CS selecting the write signal DDS;
database signal SDA for communication I2C at RTC;
clock signal SCL for I2C communication at RTC;
signal Power switch of auto maintenance switch on (active on 1);
signal I_out_set rectangle with variable fill factor for the generator PWM for command output current level;
clock signal DDS_CLOCK for synthesizer (DDS);
signal LED from flagging operation through the intermittent lighting up of the led;
read signal Audio from memory and filtered from the generator PWM out of pin 30 of the microprocessor M;
signal Backlight coming out of the pin 1 of the microprocessor M and to light backlight display device DA
input signals in the microprocessor M:
signal (l_meas) measurement of output current that comes in the form of alternative and is numeric filtered in the microprocessor (M) and measured;
signal (Bat_meas) of constant voltage divided from the supply voltage for battery monitoring;
signal (Key_enter) given by the on/off switch to turn off the device
signal (Ref_2048) of the reference voltage for measurement current

Having in view of requirements of communication with a computer, it can be used, for example, an ARM Cortex-M3 microprocessor that does not require programming, BOOT LOADER for loading making it via USB like loading a memory stick directly under Windows. This microprocessor must have communication systems such as I2C and SPI and miniature capsule (LQFP48).

Sinusoidal signal generated by DDS enters the digital amplifier blocks BAD and audio BA with an adjustable constant current which can vary from 1 to 400 mA but output will have only one preset value. Both the frequency and current are continuously controlled by M. The outputs of the amplification filtered blocks are applied to the relevant terminals of the device D that is connected functionally to coils 8, 9 and respectively, 19 based on what mouth guards A, C, and/ or F are used at the time.

Integrated circuit of the synthesizer DDS, shown in FIG. 24, has two registry of frequency R_{freq} and two registry of phase Rₚₕₐₛₑ where you can write two frequencies and two phases in a system of interface SPI. In this case, set only the frequency register R_{freq}. Sinus table is in SIN ROM and his running will be done at the written frequency of the clock generator RTC gave by the microprocessor M. For example, using a clock generator integrated RTC 7W-25,000 MBAT of 25 MHz or clock signal is obtained with processor M by splitting the quartz crystal of 12 MHz through 2¹⁰ and get 11718.75 Hz which is the reference clock signal of DDS synthesizer.

Frequency resolution of the output current is the ratio between clock frequency and the frequency register 2²⁸

Rez=11718.75/2²⁸=0.000043655 Hz.

The sinus block generator PMW consists of some modulators involving current reaction and limitation of PWM-signal value. It uses the system to compare a triangular high-frequency signal (120 Khz) with sinusoidal signal generated by DDS, such that the input signal is converted to a rectangular symmetrically signal. This symmetrical signal is amplified and added with the current reaction signal and applied to block digital amplifier BDA. Thus it ensures the voltage reference for the attachment of reference sinusoidal signal in digital amplifier blocks BAD and respectively audio BA and in microprocessor M in the measurement.

In the block digital amplifier BAD with current reaction, the input signal from the sinus block generator PMW is applied to pin 8 (IN) reference signal pin 10 (BIAS). Output current passes through a resistance reaction and measure R131. The voltage produced is amplified of block BA and represents the current value of output for measurement (I_MEAS) and in the meantime added through R125 with input signal, creating the current reaction. The current output is obtained from out1 and out 2 by filtering passes down with filters and l101, c102, l121 and c127.

BAD block can contain a linear amplifier, but its efficiency is 56% maximum when the output voltage has excursion equal to the supply voltage, but in this case, where the resistance of the mouth guards has various values, it follows that the efficiency is between 0% and 56%, which is not allowed because it cannot ensure the autonomy of operation and evacuation.

Such it uses linear amplifier version in class D (switch), for example the MAX9768. The power requirement is 3.6W RMS in output.

In the audio amplifier block BA audio signal resulting from the microprocessor M is applied at the input block amplifier BA, where is boosted 4.7 times and plugged into the speaker through the terminals w201 and w202.

Having the low supply voltage (3.7V) choose for block BA a small voltage in topology BTL low power. You can choose for example integrated circuit LM4864.

The real time clock RTC is made with an usual integrated circuit work, e.g. DS 1337 and who communicate with the microprocessor M and is powered permanently with 3 volt auxiliary battery which ensures the independence of at least two years. Setting the date and time and reading is done on the microprocessor M.

Memory block WB has in composing an integrated circuit, i.e. 24 LC1024 where you can save 1024 Kbyt. The audio file is saved from the microprocessor M and converted into PWM signal that by filtering represents memorized audio signal. Choose a memory of 1024 Kbps because you need to load the audio file in it that has minimum 200 Kbytes and at the same time it must be memorized treatment history and any information. For example, you can use memory 25LC1024.

The on/off switch block BOP contains a floor switch l1 and a floor loading l2. The electronic floor switch l1 is made with mosfet type U205, transistor of auto maintenance q202 and adjacent resistors. The pressing starting stop by D209, a resistance R223 that polarize their circuit grid U205 opens coupling the battery to device D.

After resetting the first thing microprocessor does is signal generation Power that opens transistor q202 and auto maintenance open the circuit U205.

To stop, by pressing switched off the voltage from pin 33 of microprocessor M is cancelled by a diode D102, which means off to signal processor M which makes life-saving functions of the parameters and then cancel the signal Power and the device stops.

Floor loading l2 is achieved by clock generator PWM U202, semi bridge of mosfet U203, voltage regulator U207 and current limiting circuit 0204.

The introduction of charger is supplied clock generator PWM U202 and creates signal LOADING. At exit filter L202 4.2 V voltage occurs which is controlled via the generator PMW U207 modulating created signal PWM. Microprocessor M being supplied with this voltage starts and detects the signal LOADING.

Then open the electronic switch CE and is coupled the accumulator to charging.

Load current is measured on the resistant group R205, R211 and R214 and the generator PMW U204 and adjusted by PWM modulation at 2 A value by reducing the voltage of 4.2 V.

Under the current of 2 A battery voltage grows and at achieving the value of 4.2 Volt, voltage regulator takes signal modulation PWM to maintain voltage 4.2 V, which is reflected in the reduction of load current up to 0.

The power supply ALIM incorporates a source of 14 volts which is a Boost Converter type and is made with an integrated circuit U201 and transistor Q201. The output power is limited to 5 W by choosing the value of sense resistances, R206, R209 and R210. ALIM block also contains a 3.3 volt source performed with an integrated circuit, for example MCP1804 which has the minimum input voltage 3.4volts.

Basically, it uses the supply voltage of a Lithium-Ion cells to ease loading system in case you place several batteries in parallel and not in series since then should be balancing circuit. ALIM block must be a source of constant current limiting when the voltage reaches 4.2V. Battery capacity is determined according to the maximum output power, efficiency and autonomy of operation. For global efficiency of 80% and a minimum voltage of the power supply 3.5 V from calculations resulting a energy of 5 Wh.

Must be ensured a stream of 1.42 Ah. For a greater autonomy of two hours you use two batteries connected in parallel with each capacity 2 Ah.

a. for 3.3 V supply is choose a linear source LD (with little voltage drop at saturation), because the input voltage is between 3.5 and 4.2 V (do not justify source commutation).

b. to supply power floor it takes 14 V and choose Variant Boost Converter, for example MAX668.

The results of tests carried out on patients

The inventor's personal research has focused on two distinct directions: a system innovation of bone integration implants zirconium oxide-topic presented in detail in the presentation of the previous state of the art and the influence of electromagnetic field on the bone tissue, we will develop in detail in the chapter. The purpose of the research was finding an adjuvant therapies in treatment of periodontal maintenance so patients diagnosed with this disease to preserve natural as much hands-on time.

Based on a relatively diverse literature in an area insufficiently explored, it has managed to identify and isolate frequency electromagnetic with regenerating properties. The description of the principle of operation of the device (D) as well as biocompatibility studies are described in a previous chapter.

In the area of dentistry kit and its action have been tested in field of periodontal and endo donthosis pathology and periimplantation while increasing the healing process respectively in osteointegration implantation therapy. Clinical trials took place both at the private clinic in collaboration with clinicians in the country and abroad.

In parodonthology field, clinical study was extended for a period of 24 months and included a batch of 20 patients diagnosed with acute/chronic periodontitis residential generalized or localized, aged 29 years and 60 year-old clinically healthy undiagnosed metabolic or systemic diseases that find their reflection in oral area as well as patients who do not follow any curative treatment. Select patients having a history periodontal that does not include specific treatment, only sporadic painting professional, including scaling and brushing.

After general dental status accompanied by radiological examinations and laboratory tests, patients were carried out initially with periodontal status setting plaque indices and parodontometry.

Taking into account the complex nature of periodontal disease were determined the main directions of treatment stated the three main phases of treatment.

Treatment of patients was limited to local character of periodontal disease by following the following directions:

1. Antimicrobial treatment;

2. Surgical treatment;

3. Treatment of occlusal equilibration;

4. Treatment of structural and functional rehabilitation by marginal periodontitis bio reactivation;

Once established these general directions of treatment they have undergone small changes depending on the status of the further evolution of the disease as well as time and material possibilities of patients.

The initial treatment was focused on intervention on the acute complications of marginal chronic parodonthosis by gingival scaling and debridement, scaling ultrasonic manual supra and sub gum complete sterilization using a Nd: YAG LASER diode with a wavelength of 20 Hz, as well as the Suppression of infectious and inflammatory processes maintained by this root rests stranded. A very important role we have attributed to awareness patients' about the stage of the disease, they presented, and instruction on cleaning procedures to be carried out at home by brushing and utility assets.

To assess the level of oral hygiene has been used oral hygiene Index-Oral Hygiene Index-OHI-what is a combined index, composed of calculus of soft deposits-index-index DI and the detritus of tartar index-Cl-. Each of these two indices is numerically determined on the basis of quantity of soft deposits, namely Tartar, evidenced on the vestibular and oral surface of teeth, each of the arches being divided into three segments. For each of the six segments is taken into account the tooth with the greatest value for the segment in question.

Criteria for evaluation of soft deposits-DI-are:

Score 0-hard or soft deposits colorations absent;

Score 1-soft Deposits present in the gingival third (but not much) or extrinsic colorations without soft deposits;

Score 2-soft Deposits present between one third and two thirds of the surface of the tooth;

Score 3-soft Deposits present on a stretch of more than two-thirds of the dental surface.

Assessment criteria for the classification of the deposits of tartar (CI) are:

Score 0-the Tartar deposits absent;

Score 1-Deposits of supra gum calculus in gingival third-but no more;

Score 2-Deposits of supra gingival Tartar present between one third and two thirds of the sub gingival tooth surface or scale in the form of islands along the cervical area, or both forms.

Score 3-Deposits of supra gingival Tartar present on two-thirds of a tooth surface sub gingival Tartar in the form of continuous tape along the cervical, or both forms.

After the assessment scores for soft and Tartar deposits, calculated value of the OHI.

DI=the sum of the individual values/number of segments analyzed

CI=the sum of the individual values/number of segments analyzed

Calculation formula: OHI=DI+CI.

Patients were recalled to regular checks with a frequency weekly during the first month and then twice a month and help/reinstructed on cleaning techniques, revealing areas where we must insist with sanitizing so each has managed to obtain an index OHI between 1 and 2. Only from this moment could move forward towards the secondary phase of treatment.

Corrective treatment consists in restructuring procedures, re equilibration dental-occlusion through sanding occlusal detection and removal of iatrogenic factors, like odontal obstruction not-adapted from dental Crown contour, crowns with a rational adaptation to the gingival contour, restoring dental morphology affected by caries, prosthetics restoration of parodontothic teeth immobilization with fiberglass strips where they require it.

In this phase it was applied the treatment of bio reactivation with electro-magnetic field of very low frequency emitted by the device D through the coil 9 or 15 arranged in the applicator E.

Applicator E electromagnetic field CEM has a U shape that comprises between the two poles the area that require regeneration. The distance between the poles is variable depending on the thickness of the arch in the area of application, ranging between 15 and 21 mm. The distance that acts CEM is the maximum length of the applicator. Applicator E in turn is placed and restrained on a internal mouth guard A or B or are used directly on F for each patient, the area requiring treatment. In this way there is the certainty that the applicator E will act over the desired area without being able to be mobilized by the patient.

In order to accomplish the mouth guards A or B or F for support the applicator E, fingerprinting, arches jaw models casting plaster work and making mouth guards A or B or F of thermoplastic foil about 2 mm thick. Applicators E are bonded and secured on area that requires treatment and connected through a cable to the device D transmitter of CEM. On an archway can be installed only two (mouth guard B) or three (mouth guard A) such applicators E simultaneously due to mouth guard very limited space of the oral cavity, including between their poles a segment of maximum 3 teeth. The patient is instructed in practical way to be applied the mouth guard A or B or F on the dental field, how hygienic, how you have connected the kit, the number of exposure to CEM.

CEM exposure is done in sessions of 1-2 hours each in an initial number of minimum 30 sessions, during which the patient is unable to speak, eat or drink but can perform simple activities in the comfort of home. The sessions are recommended to be performed daily for a maximum efficiency of treatment or with an interval of not more than 7 days between them.

After applying the previous two phases of treatment it follows the treatment to maintain obtained results through individualized procedures aimed at preventing reinfection, installation of new morphological disorders and dysfunctions and consequently the relapse. Because exposure to EMF works through a mechanism of stimulation of growth factors at the tissue level, we recommend treatment with the dental kit, according to the invention, in the end phase of treatment too in prophylactic scope once a year.

The treatments carried out on periodontitis, no matter how difficult, costly and correctly executed, are not followed by lasting results over time if you don't establish a rigorous inspection program. It follows the oral hygiene status: the most important element of maintaining therapeutic outcomes, prevention of relapses by gingival scaling and debridement, treatment of tooth decay, occurred later. Screening for patients and periodic examination is the rule: in the first year on a quarterly basis, to six months in the second year after treatment and yearly or anytime you need to install the relapse of the disease.

Another embodiment of the treatment is the following:

The number of sessions is determined by your dentist following radiological analysis of patient and clinical examination for determining the mobility of the teeth, and can vary from 60 hours of treatment up to 200 hours of treatment for each dental arcade.

Medical device software allows you to activate by your dentist the generator that remains locked until doctor dentist recommendation through software installed in the computer of logistics officer of the treatment is carried out in medical grant number of the initial hours of the electromagnetic therapy. The dentist decides, for each individual patient, time of visit and, at that point, your dentist will decide, based on the patient's examination if required overtime of electromagnetic therapy or patient must return the generator. The duration of a session is 2 hours and it is recommended 3 sessions a week to two days between them for each of the dental arches subjected to regeneration. In the following it will exhibit some clinical cases from inventor study and through the kindness of some clinicians researchers from Romania and abroad.

The action of EMF emitted by the kit, according to the invention, in Periodontology

1. Patient S.B. presented at clinic accused acute pain and mobility at tooth 3.5. Periodontal probing showed existence of periodontal pockets of 7 mm on mezial face and 5 mm on the front distal. The patient was applied Protocol explained previously and has been recommended in the first phase the restoration of the upper jaw and the application (CEM) in quadrants 2 and 3 in a total of 60 sessions-30 sessions for quadrant 3 and 30 sessions for quadrant 2.

Initial Radiological Image is Shown in FIG. 29

The patient opted for metal-ceramic restorations and for exposure to CEM stages, first in the square on 3 and then for the rest of the teeth that have made recommendations. The initial and intermediate situation in applying the proposed treatment plan has been registered in the computerized program specially created for the accounting and recording of the evolution of each case.

Graph records on an axis, the degree of gingival retraction, the degree of mobility and depth of the periodontal bags, measured on the vestibular and oral faces of teeth to be treated.

The patient lies still in the process of being treatment. After 30 sessions of the CEM applied to his level 3.5, parodontometry showed decreased with 2 mm of the periodontal bag both front mezial and distal, and radiological image showed bone densification through the appearance of new bone trabeculae and decrease periodontal tooth space to 3.5.

Radiological image after applying partial treatment plan proposed, namely prosthetic restorations in quadrants 2 and 3 and application (EMF) 30 sessions to 3.5 in the tooth as shown is FIG. 30.

Images of the graph of the evolution of treatment, here emphasizing V and L face measurements of 3.5 are played in FIG. 30.

2. Patient M.L. aged 55 years, was presented at clinic accused acute pain and high mobility at tooth 4.7. Clinical examination showed presence of periodontal pockets of 7 mm on the front mezial and mobility grade 1. Radiological examination confirmed the presence of this Periodontal pockets. The patient was recommended treatment for exposure to CEM in the affected area in 30 sessions.

Initial Radiological Image is Illustrated in FIG. 31.

Radiological image surprised after treatment; illustrated in FIG. 32 distinguishes bone densification. Clinical parodonthometry showed a decrease in periodontal pocket at 3.5 mm and degree of mobility has been reduced significantly towards 0.

3. Patient aged 45 years it has been presented to the clinic for routine control of implants. Clinical examination showed gingival mucosa bleeding on palpation to 2.6. Radiological exam was noticed increased radio transparence with bone implants from dial-area 2. As the treatment plan we have recommended strict cleaning and exposure to CEM.

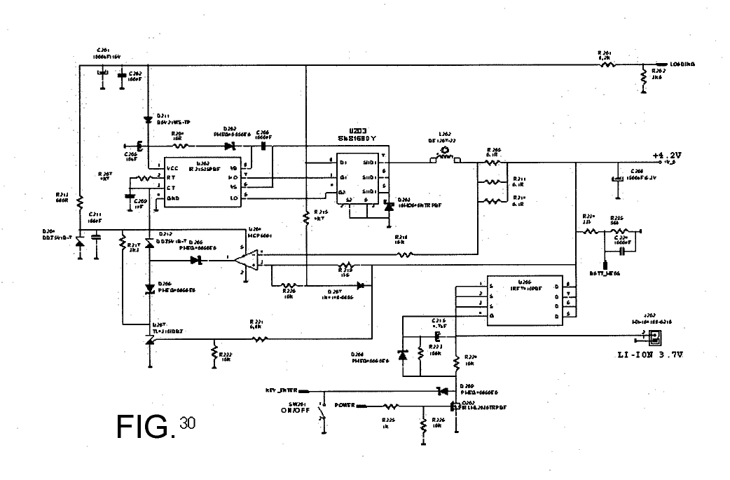

Initial Radiological Image is Illustrated in FIG. 33

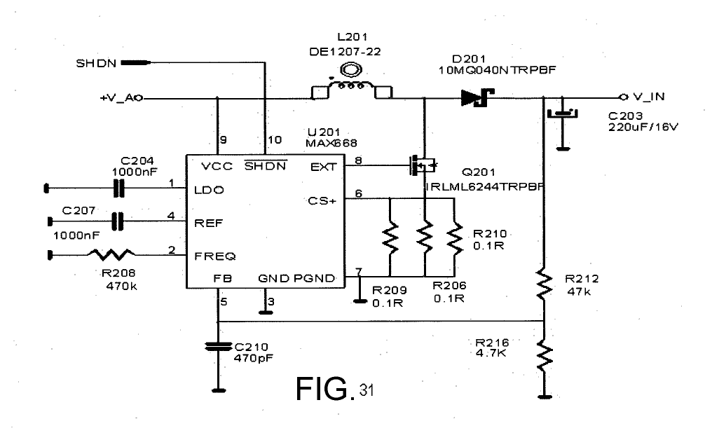

Radiological image after the 30 sessions is presented in FIG. 34. It is observed the reduction of the space periimplantar with apositis bone minor vertically. Clinical signs specific, mucositis were fined. The patient was recommended to resume meetings of the CEM exposure after 6 months with the preventive character.

The Action of CEM Emitted by Kit According to the Invention, in ENDODONTICS

To illustrate more eloquent action dental kit delivered by CEM in accordance with the invention, we expose in the following fragments from a clinical trial conducted at the Faculty of Dentistry of Titu Maiorescu Univ. Bucharest, in 2014.

From the medical point of view, the existence of a Granuloma chronic apical represents a danger to the whole body causing a disease outbreak. In most cases, patients presenting chronic periapicale do not reach the dentist only after injuries natural granuloma.

Granuloma chronic apical require either conservative treatment by successive application of antiseptics and calcium hydroxide, or surgical treatment. By applying conservative treatment with calcium hydroxide and antiseptic, cannot predict the long-term success of the treatment or failure rate.

Dental Kit is a medical device used in the treatment of periapicale pathology, which consists in the application of -low-frequency electromagnetic field emitted from the applicator E placed on a mouth guard A or B or total applicator F, for a minimum of 15 sessions. It is a painless treatment, stem cells stimulating of the bone and dentine depth, dental pulp, used for prophylactic and bone regeneration and gingival. This procedure is indicated for the treatment of bone and resorbtion of apical lesions.

Materials and Method

The tooth is 2.1 which presents a Granuloma apical of 1.5 mm in diameter, diagnosed during a routine check. The tooth was treated in the channel by mechanical and chemical properties, after which was applied treatment with dental Kit according to the invention. It is very important to be minimized any microbial activity prior to use the dental kit according to the invention. CEM exposure took place in 15 consecutive sessions of 120 min per session, followed by the endodonthic obstruction. Evolution of treatment was followed by x-ray examination.

### Results

Pulsed electro-magnetic field of low frequency generated by applicator E stimulated cells osteoblast and inhibit osteoclasts cells thus resulting in complete healing periapical. The new bone tissue have the same aspect and feel as well as healthy bone tissue radiological from proximity.

Treatment with dental Kit is painless, needle, periapical healing occurring faster. During treatment, the patient did not give signs of discomfort or pain in deglutition of the temporo-mandibullar joints TMJ pathologies. Dental kit is easy and safe to use without a danger under his proper use.”

The patient whose case was presented in the study provided us recently, a new x-ray done within the program for tracking the evolution of treatment carried out a year ago. The complete healing of the area periapicale and not any relapses.

2. Patient I.D. aged 38 years, was presented the clinic with the purpose of prosthetic rehabilitation from the dial 1. Initial radiological examination revealed the presence of periapicale injuries to teeth 1.3 and 1.4. With the aim of prosthetic rehabilitation, endodonthic retreatment was recommended to completely and correctly all the teeth that will be made up of elements of the future support bridges, complemented by exposure to CEM for quick and complete healing.

The patient agreed treatment plan proposed and we have remade endodonthic treatments. Along with dental Kit and sessions based on the favorable evolution of healing apical lesions existing took place and morpho-functional rehabilitation through a metal-ceramic bridge.

3) Action CEM by dental Kit according to the invention to a patient with gingival and bone graft:

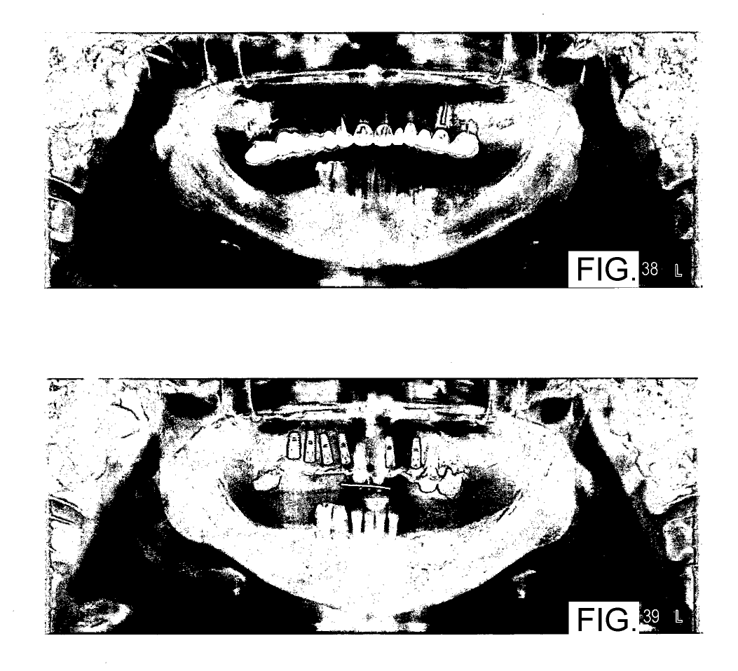

Initial Radiological Image is Illustrated in FIG. 38

CEM exposure is carried out in meetings of 2 hours each in an initial number of not less than 30 meetings, during which the patient is unable to speak, eat or drink but can perform simple tasks in the comfort of home. It is recommended that the meetings are carried out daily for maximum efficiency of treatment or with an interval of not more than 7 days between them.

Clinical case. The patient presents a mandible cyst which is surgically remove and produces a very large bone defect which under normal conditions is not remake

It is recommended the patient's treatment with electromagnetic field exposure daily for 2 months.

Radiological image after treatment is viewed in FIG. 39, and healing through the total rebuild of the bone mass in the area of the defect after the surgery. Clinical integration was noticed and graft-free gum applied to close the defect during the postoperative period.

1. Dental kit that comprises an mouth guard (F) with multiple work-points, a material (12) consisting of a polymer and a device (D) generating an electromagnetic field which generates, inside the mouth guard, an uniform field with an induction field ranging between 0.7 . . . 0.9 mT, with a variation ranging between 5 . . . 10%, in the area subjected to the electromagnetic field, on a distance of 1 cm around the same point, and on a volume as high as possible in the areas where the tooth root or the implant is to be positioned, produced by a current of 200 mA, with a frequency ranging between 7 and 8Hz, preferably 7.69 Hz, the field lines being perpendicular on the target-tissue, which are placed in a box, and where
the mouth guard (F) that acts on the entire arcade consists of a metal curved body (14), that follows the anatomical shape of the jaw, comprising two walls parallel to each other (i″ and j″), curved exterior and respectively, interior, bonded among themselves, upper or lower, depending on the use, on the upper or lower jaw of a superior wall (k″), the body (14) forming the metallic core of a coil (15) made of copper wire, frontal, the coil (15) having two electric conductors (16 and 17), frontal, through which it is carried out electrical connection with the device (D), on the outside, the coil (15) being covered by a layer (18) made of polyethylene or of the material (12); and in that
the material (12) is in the form of the sandwich being subsequently a body (11) of a personalized internal mouth guard (C) that follows the anatomical shape of the jaw; and in which
the device (D) is a generator of sinusoidal low frequency current in 2 Hz-25 Hz range, with the level of output current 200 mA, maximum output voltage 24V, and is built around a microprocessor (M) which generates the signals of low frequency current which generates an electromagnetic field of uniform induction intensity on a distance of 1 cm around the same point on a larger volume in areas that would be positioned dental root implant, of 0.75 mT-0.9 mT, and a direct digital synthesizer (DDS) adapted to directly generate sinusoidal signal precisely with a variation from 2 to 50 Hz of high precision and stability led by the processor (M), the device (D) containing a processing block (BP), the synthesizer (DDS), a digital amplifier block (BAD) with current reaction, an audio amplifier block (BA), a clock block (RTC) for real time, a block of memory (BM), an on/off switch block (BOP) and a power supply (POWER).

2. Dental kit, according to claim 1, characterized in that, a body (11) of the personalized mouth guard (C) has some sockets (1″) placed in front of the teeth of the oral cavity, and if there are any missing teeth in their own front, between two sockets (1″) surrounding free space, is placed a blunt (m″) touching the gums.

3. Dental kit, according to claim 1, characterized in that, the personalized mouth (C) is made and fixed by gluing to the wall (k″) of the mouth guard (F) with multiple work-points.

4. (canceled)

5. Dental kit, according to claim 1, characterized in that the material (12) in the form of a sandwich consists of a polymer, in which mineral granules are embedded with crystalline structure or substance or, preferably, natural, consisting of topaz, jade, calcium carbonate, octocalcic phosphate, silicates, quartz crystals, salt or natural ground, volcanic rocks, dust from animal horns or granules of zirconium oxide alone or in a mixture, the size of the particle being of 20 . . . 150 nm

6.-9. (canceled)

10. Dental kit, according to the claim 1, characterized in that the coil (15) is uniform wired, wire near wire, so as to be a uniform induction electromagnetic field in the outer edges of the wings parallel to each other, and when the current is raised to 400 mA, to determine in the continuance of the wings a volume where the field is uniform, in the depth of the bone.

11. Dental kit, according to the claim 1, characterized in that the mouth guard (F) is made of two separate semi-mouth guards (F1 and F2) which together cover the entire jaw, each can be a semi-arch, each semi- mouth guard (F1 and F2) having frontal a coil (15′), respectively (15″), two frontal electric conductors, (16′ and 17′) and respectively (16″ and 17″), through which it is carried out with the electrical connection with the device (D), thus, the electromagnetic field generated by each (F1 or F2) or both (F1 and F2) has a high uniformity, with a variance of less than 8%, on the whole area being treated.

12. Dental kit, according to claim 1, characterized in that the processing block (BP), consisting of the microprocessor (M), a display device (DA) and some connection jacks (USB), generates through microprocessor (M) the following signals:
clock signal (SCK) for serial communication;
data signal (SDATA) for serial communication;
signal (CSD) to pin nr 37, selecting the display device (DA) during communication;
signal (MEM CIP SELECT) selecting memory during communication with it;
signal (SO) selecting read-write memory;
signal (DDS_CS) selecting the write signal DDS;
database signal (SDA) for communication I2C (at RTC);
clock signal (SCL) for I2C communication (at RTC);
switch signal (Power) of auto maintenance on switch (active on 1);
signal (I_out_set) rectangle with variable fill factor for the generator (PWM) for command output current level;
clock signal (DDS_CLOCK) for synthesizer (DDS);
signal (LED) from flagging operation through the intermittent lighting up of the led;
read signal (Audio) from memory and filtered from the generator (PWM) out of pin 30 of the microprocessor (M);
signal Backlight coming out of the pin 1 of the microprocessor (M) and to light backlight display device (DA)
input signals in the microprocessor (M):
signal (I_meas) measurement of output current that comes in the form of alternative and is numeric filtered in the microprocessor (M) and measured;
signal (Bat_meas) of constant voltage divided from the supply voltage for battery monitoring;
signal (Key_enter) given by the on/off switch to turn off the device
signal (Ref_2048) of the reference voltage for measurement current

13. Dental kit, according to claim 1, characterized in that the synthesizer (DDS) contains a sinusoidal signal generator (PMW) made with modulators which incorporates current reaction and limiting the signal (PMW).

14. Dental the kit, according to the claim 1, characterized in that the digital amplifier block (BAD) with current reaction, contains a linear amplifier of switching class D.

15. Dental kit, according to the claim 1, characterized in that the audio amplifier block (BA) is an amplifier of low-voltage in power topology BTL of low voltage .

16. Dental kit, according to claim 1, characterized in that the memory block (BM) has an integrated circuit having a memory of 1024 Kbytes for uploading audio file and memory history of treatments and any information.
